(19) United States
(12) Patent Application Publication
(10) Pub. No.: US 2006/0035294 A1
(43) Pub. Date: Feb. 16, 2006

(54) Use of the protein maba (fabg1) of mycobacterium tuberculosis for designing and screening antibiotics

(76) Inventors: Annaik Quemard, Montgiscard (FR); Gilles Labesse, Montpellier (FR); Mamadou Daffe, Toulouse (FR); Hedia Marrakchi, Toulouse (FR); Dominique Douguet, Montpellier (FR); Martin Cohen-Gonsaud, Nimes (FR); Stephanie Ducasse, Toulouse (FR)

Correspondence Address: YOUNG & THOMPSON, 745 SOUTH 23RD STREET, 2ND FLOOR, ARLINGTON, VA, 22202, US

(21) Appl. No.: 10/503,939

(22) PCT Filed: Mar. 28, 2003
(86) PCT No.: PCT/FR03/00990

§ 371 Date: Jun. 27, 2005

(30) Foreign Application Priority Data
Mar. 29, 2002 (FR) 02/04018

(51) Int. Cl. G01N 33/554; G06F 19/00; C12N 1/21; C12N 15/74; C07K 14/35

(52) U.S. Cl. 435/7.32; 530/350; 435/69.3; 435/252.3; 435/320.1; 702/19

(57) ABSTRACT

The invention especially relates to the protein MabA, also named protein FabG1, which is recombinant in a purified form, or the recombinant proteins derived from the protein MabA by mutation of at least one amino acid. The invention also relates to the uses of said proteins and to the crystallographic co-ordinates thereof, in terms of the implementation of methods for designing and screening ligands of said proteins, and advantageously, ligands inhibiting the enzymatic activity of said proteins.

The main subject of the present invention is the use of the protein MabA, and derived proteins, and more particularly the crystallographic co-ordinates of these proteins, within the framework of the implementation of methods for designing and screening ligands of these proteins, and advantageously ligands inhibiting the enzymatic activity of these proteins, namely antibiotics capable of being used within the framework of the treatment of mycobacteriosis.

Tuberculosis is one of the major causes of mortality by a single infectious agent, Mycobacterium tuberculosis. Moreover, for about fifteen years, there has been a recrudescence of this disease in industrialized countries. This phenomenon is linked in part to the appearance of antiobiotic-resistent strains of Mycobacterium tuberculosis. Thus, the design of new antituberculous-medicaments has become a declared priority of the Word Health Organization.

The targets of the antituberculous antibiotics currently used in clinics form-part of biosynthesis metabolisms of components of the envelope of Mycobacterium tuberculosis. In particular, the target of isoniazid (INH), a 1st-line antituberculous agent, is involved in the synthesis of very long-chain fatty acids (C60-C90), the mycolic acids. Isoniazid inhibits the activity of the protein InhA, which forms part of an enzyme complex, FAS-II, the function of which is to produce, by successive elongation cycles, long-chain fatty acids (C18-C32), precursors of the mycolic acids. InhA, a 2-trans-enoyl-ACP reductase, catalyzes the 4th stage of an elongation cycle, which comprises 4 stages. INH is a pro-drug which forms, with the coenzyme of InhA, NADH, an inhibiting adduct, INI-NAD(H). FAS-II comprises at least 3 other main enzymes, the latter therefore representing potential targets for novel antibiotics. The protein MabA catalyzes the 2nd stage of the cycle.

The present invention provides methods for the design of antibiotics for the treatment of mycobacterial infections, in particular tuberculosis. This invention deals with the mabA (fabG1, Rv1483) gene of Mycobacterium tuberculosis, the product of this gene, the protein MabA (FabG1), as well as with the material and methods used for the production of the protein in a large quantity, the determination of its three-dimensional structure on the atomic scale and the study of its interactions with different ligands or their effect on its enzymatic activity. The invention is based on the use of the protein MabA as a target for antibiotics; in particular, the study of the interaction of MabA with different ligands or their effect on its enzymatic activity, by different methods, is used in order to design inhibitors of the enzymatic activity of MabA.

The-present invention provides the methodological tools and material necessary for designing molecules representing potential anti-mycobacterial and antituberculous antibiotics.

The present invention proposes the biological material and the methodologies necessary for the production and purification, in large quantities, of a potential target of antituberculous antibiotics, the protein MabA. Moreover, these stages can be carried out very rapidly thanks to the overproduction of MabA provided with a poly-Histidine tag in Escherichia coli and its purification in a single stage by metal chelation chromatography, producing a protein with a high degree of purity. The quantity and quality of the purified protein make it possible to obtain reliable results during studies of enzymatic activity or binding of ligands, but also allow the crystallization of the protein in order to resolve its three-dimensional structure. The development of conditions allowing the freezing of the MabA crystals in liquid nitrogen has made it possible to obtain sets of atomic resolution data (2.05 Å compared with 2.6 Å at ambient temperature) and opens the way to better data thanks to the use of synchrotron radiation. The frozen crystalline structure has revealed the role of compounds (in particular caesium) necessary for the growth of the MabA crystals and makes it possible to envisage rational optimization of crystal growth. The screening in crystallo of “pools” of ligands can also be carried out. The quantity of protein purified is also an important criterion for carrying out high through-put screenings of combinatorial libraries (see below).

The protein MabA activity tests, and as a result the tests on inhibition by potential inhibitors, can be followed easily and rapidly by spectrophotometry, by monitoring the oxidizing of the reduction coenzyme, NADPH, at 340 nm. The inhibition constants (IC50 and Ki) and the inhibition mechanism (competitive, non-competitive, uncompetitive inhibition) for each molecule can be deduced from this. Moreover, tests on specific binding of ligands to the active site of MabA can be also carried out easily and rapidly, by spectrofluorimetry. The presence of the single Trp residue of the protein in the active site makes it possible, by excitation at 303 nm, to detect, from the variation in the fluorescence emission intensity at the emission maximum, the binding of a ligand and to deduce from this the disassociation constant (Kd). Similarly, FRET (Fluorescence Resonance Energy Transfer) experiments can be carried out in the presence of the coenzyme, NADPH, making it possible to conclude from this whether or not the ligand occupies the binding site of the NADPH (binding competition). The simplicity of these measurement methods, and the relatively low volumes that they require, will allow a miniaturization of the inhibition or ligand-binding tests, for the automatic high through-put screening of combinatorial libraries, thanks to an automatic device provided with a spectrophotometer or spectrofluorimeter.

Comparison of the structure of MabA with that of the-protein InhA, a protein of the same structural super-family (RED) and belonging to the same enzyme complex (see above), suggested an inhibiting effect of isoniazid on MabA activity and detection of the active form of isoniazid (antituberculous), the INH-NADP(H) adduct. The binding of the adduct and inhibition of the MabA activity was then confirmed experimentally. Similarly, thanks to a strong structural similarity with other proteins, which have been or will be crystallized with ligands (for example, steroid derivatives, co-crystallized with steroid dehydrogenases), the rational design of potential MabA inhibitors can be carried out rapidly.

The protein MabA is of particular interest as a target of anti-mycobacterial antibiotics. In fact, it forms part of the same enzymatic system as the protein InhA, target of the 1st-line antituberculous medicament, isoniazid. On the other hand, up to now, no protein homologous to MabA has been detected in animal cells. Moreover, comparison with the homologous proteins found in bacteria or plants has highlighted particular properties of MabA, which are linked to its function, since it uses long chain substrates. These characteristics are reflected in the structure of its active site, which makes it possible to envisage the design of inhibitors specific to MabA (in particular, in terms of size and hydrophobic character), and therefore of narrow-spectrum antibiotics. These different points provide MabA with criteria for pharmacological credibility.

Thus, the main object of the present invention is:

research into and design of medicaments effective against opportunist mycobacterial infections (M. avium, M. kansasii, M. fortuitum, M. chelonae etc.) presenting problems in hospitals (sterilization of medical instruments), and in the case of human immuno-deficiency (AIDS, administration of immunosuppressors during a graft, in the case of cancers etc.).

research into and design of medicaments effective against tuberculous infections, in particular medicaments which are effective on the strains of M. tuberculosis resistant to the antibiotics currently used in antituberculous therapy, and which are propagated in populations at risk (prison environment, economically disadvantaged environments etc.).

research into and design of medicaments effective against other bacterial infections, by taking proteins homologous to MabA as-molecular targets.

A main subject of the present invention is the protein MabA, also called protein FabG1, recombinant in-the purified form, or the recombinant proteins derived from the protein MabA by mutation of one or more amino acids, said derived proteins being in purified form, and having an NADPH-dependent β-ketoacyl reductase activity.

A more particular subject of the invention is the purified recombinant protein MabA, said protein being a protein of mycobacteria, such as Mycobacterium tuberculosis, and more particularly M. tuberculosis strain H37Rv.

A subject of the invention is also the recombinant protein MabA or the abovementioned derived recombinant proteins, in purified form, as obtained by transformation of strains of E. coli with a plasmid containing a sequence comprising the gene coding for the protein MabA, or comprising a sequence coding for a protein derived from MabA as defined above, followed by a purification stage during which:

the abovementioned recombinant E. coli bacteria are washed in a washing buffer, then taken up in a lysis buffer, and lysed by a freeze/thaw cycle in the presence of protease inhibitors and lysozyme,

after treatment by DNAse I and RNAse A, in the presence of MgCl₂, and centrifugation, the lysis supernatant of the bacteria obtained in the preceding stage, to which 10% (v/v) of glycerol, or 400 μM of NADP⁺ is added, is applied to an Ni-NTA agarose resin column,

after several washings with 5 mM buffer then 50 mM imidazole, the protein MabA, or the derived protein, is eluted with the elution buffer.

According to an embodiment of the invention, the recombinant protein MabA or the abovementioned derived recombinant proteins, in purified form, are obtained according to the process described above in which the different bacteria washing, lysis, washing, and elution buffers are the following:

bacteria washing buffer: 10 mM potassium phosphate, pH 7.8,

lysis buffer: 50 mM potassium phosphate, pH 7.8 containing 500 mM of NaCl, 5 mM of imidazole,

washing buffer: 50 mM potassium phosphate, pH 7.8 containing 500 mM of NaCl, 5 and 50 mM of imidazole,

elution buffer: 50 mM potassium phosphate, pH 7.8 containing 500 mM of NaCl, and 175 mM of imidazole.

Advantageously, the proteins obtained using the :abovementioned buffers are used within the framework of enzymatic kinetic studies for the screening of ligands according to the methods described hereafter.

According to another embodiment of the invention, the recombinant protein MabA or the abovementioned derived recombinant proteins, in purified form, are obtained according to the process described above in which the different bacteria washing, lysis, washing, and elution buffers are the following:

bacteria washing buffer: Tris 10mM, pH 8.0,

lysis buffer:
50 mM Tris buffer, pH 8.0, supplemented with 300 mM LiSO₄ and 5 mM imidazole;
or 50 mM Tris buffer, pH 8.0, supplemented with 300 mM KCl and 5 mM imidazole,

washing buffer:
50 mM Tris buffer, pH 8.0, supplemented with 300 mM LiSO4 and 5 or 50 mM imidazole,
or 50 mM Tris buffer, pH 8.0, supplemented with 300 mM KCl and 5 or 50 mM imidazole.

elution buffer:
20 mM MES buffer, pH 6.4, LiSO4 300 mM and 175-750 mM imidazole;
or 20 mM PIPES buffer, pH 8.0, supplemented with 300 mM KCl and 175-750 mM imidazole,

1 mM DTT being added to these buffers in the case of the wild-type protein MabA.

Advantageously, the proteins obtained using the abovementioned buffers are used within the framework of crystallography studies for designing and screening ligands according to the methods described hereafter.

The invention also relates to the abovementioned proteins derived from the abovementioned protein MabA, and characterized in that they correspond to the protein MabA the amino acid sequence SEQ ID NO: 1 of which is the following:

MTATATEGAK PPFVSRSVLV TGGNRGIGLA IAQRLAADGH KVAVTHRGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which the cysteine in position 60 is replaced by a valine residue, and/or the glycine in position 139 is replaced by an alanine or a serine, and/or the serine in position 144 is replaced by a leucine residue.

A more particular subject of the invention is therefore the protein derived from the protein MabA as defined above, and characterized in that it corresponds to the protein MabA in which the cysteine in position 60 is replaced by a valine residue, said derived protein, also called C(60)V, corresponding to the following sequence SEQ ID NO 3:

MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

A more particular subject of the invention is therefore also the protein derived from the protein MabA as defined above, and characterized in that it corresponds to the protein MabA in which the serine in position 144 is replaced by a leucine residue, said derived protein, also called S(144)L, corresponding to the following sequence SEQ ID NO 5:

MTATATEGAK PPFVSRSVLV TGGNRGIGLA IAQRLAADGH KVAVTHRGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

A more particular subject of the invention is therefore also the protein derived from the protein MabA as defined above, and characterized in that it corresponds to the protein MabA in which the cysteine in position 60 is replaced by a valine residue, and the serine in position 144 is replaced by a leucine residue, said derived protein, also called C(60)V/S(144)L, corresponding to the following sequence SEQ ID NO 7:

MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

A more particular subject of the invention is also the protein derived from the protein MabA as defined above, and characterized in that it corresponds to the protein MabA in which the cysteine in position 60 is replaced by a valine residue, the glycine in position 139 is replaced by an alanine or a serine, and the serine in position 144 is replaced by a leucine residue, said derived protein, also called C(60)V/G(139)[A or S]/S(144)L, corresponding-to the following sequence SEQ ID NO 8:

MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIXS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X represents A or S.

The invention also relates to the protein MabA corresponding to the sequence SEQ ID NO: 1, or the proteins derived from the protein MabA defined above, such as the derived proteins corresponding to the sequences SEQ ID NO: 3, 5, 7, or 8, characterized in that they are modified such that they include one or more mutations making it possible to change the specificity of the protein NADPH to NADH.

A more particular subject of the invention is the abovementioned modified proteins MabA, corresponding to the following sequences:

the sequence SEQ ID NO: 9, corresponding to the sequence SEQ ID NO: 1 comprising the mutations N24D(or E), and/or H46D, namely the following sequence:

MTATATEGAK PPFVSRSVLV TGGX₁RGIGLA IAQRLAADGH KVAVTX₂RGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X₁ represents D or E, and X₂ represents H or D,

the sequence SEQ ID NO: 10, corresponding to the sequence SEQ ID NO: 3 comprising the mutations N24D(or E), and/or H46D, namely the following sequence:

MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X₁ represents D or E, and X₂ represents H or D,

the sequence SEQ ID NO: 11, corresponding to the sequence SEQ ID NO: 5 comprising the mutations N24D(or E), and/or H46D, namely the following sequence:

MTATATEGAK PPFVSRSVLV TGGX₁RGIGLA IAQRLAADGH KVAVTX₂RGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X₁ represents D or E, and X₂ represents H or D,

the sequence SEQ ID NO: 12, corresponding to the sequence SEQ ID NO: 7 comprising the mutations N24D(or E), and/or H46D, namely the following sequence:

MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X₁ represents D or E, and X₂ represents H or D,

the sequence SEQ ID NO: 13, corresponding to the sequence SEQ ID NO: 8 comprising the mutations N24D(or E), and/or H46D, namely the following sequence:

MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIXS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

in which X₁ represents D or E, and X₂ represents H or D.

A subject of the invention is also the protein MabA corresponding to the sequence SEQ ID NO: 1, or the proteins derived from the protein MabA defined above, such as the derived proteins corresponding to the sequences SEQ ID NO: 3, 5, 7, 8, 9, 10, 11, 12, or 13, characterized in that they are modified by insertion, on the N-terminal side, of a poly-histidine tag such as the following sequence SEQ ID NO: 14: MGSSHHHHHH SSGLVPRGSH.

A more particular subject of the invention is the abovementioned modified proteins MabA, corresponding to the following sequences:

the sequence SEQ ID NO: 15, corresponding to the combination of the sequence SEQ ID NO: 14 and the sequence SEQ ID NO: 1, namely the following sequence:

MGSSHHHHHH SSGLVPRGSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the sequence SEQ ID NO: 16, corresponding to the combination of the sequence SEQ ID NO: 14 and the sequence SEQ ID NO: 3, namely the following sequence:

MGSSHHHHHH SSGLVPRGSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the sequence SEQ ID NO: 17, corresponding to the combination of the sequence SEQ ID NO: 14 and the sequence SEQ ID NO: 5, namely the following sequence:

MGSSHHHHHH SSGLVPRGSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the sequence SEQ ID NO: 18, corresponding to the combination of the sequence SEQ ID NO: 14 and the sequence SEQ ID NO: 7, namely the following sequence:

MGSSHHHHHH SSGLVPRGSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the sequence SEQ ID NO: 19, corresponding to the combination of the sequence SEQ ID NO: 14 and the sequence SEQ ID NO: 9, namely the following sequence:

MGSSHHHHHH SSGLVPRGSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the sequence SEQ ID NO: 20, corresponding to the combination of the sequence SEQ ID NO: 14 and the sequence SEQ ID NO: 10, namely the following sequence:

MGSSHHHHHH SSGLVPRGSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the sequence SEQ ID NO: 21, corresponding to the combination of the sequence SEQ ID NO: 14 and the sequence SEQ ID NO: 11, namely the following sequence:

MGSSHHHHHH SSGLVPRGSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the sequence SEQ ID NO: 22, corresponding to the combination of the sequence SEQ ID NO: 14 and the sequence SEQ ID NO: 12, namely the following sequence:

MGSSHHHHHH SSGLVPRGSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the sequence SEQ ID NO: 23, corresponding to the combination of the sequence SEQ ID NO: 14 and the sequence SEQ ID NO: 13, namely the following sequence:

MGSSHHHHHH SSGLVPRGSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIXS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D.

A subject of the invention is also the protein MabA corresponding to the sequence SEQ ID NO: 1, or the proteins derived from the protein MabA defined above, such as the derived proteins corresponding to the sequences SEQ ID NO: 3, 5, 7, 8, 9, 10, 11, 12, or 13, having an N-terminal GSH sequence, namely the following sequences:

the following sequence SEQ ID NO: 24, corresponding to the combination of the GSH sequence and the sequence SEQ ID NO: 1,

GSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 25, corresponding to the combination of the GSH sequence and the sequence SEQ ID NO: 3,

GSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 26, corresponding to the combination of the GSH sequence and the sequence SEQ ID NO: 5,

GSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 27, corresponding to the combination of the GSH sequence and the sequence SEQ ID NO: 7,

GSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 28, corresponding to the combination of the GSH sequence and the sequence SEQ ID NO: 9,

GSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 29, corresponding to the combination of the GSH sequence and the sequence SEQ ID NO: 10,

GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 30, corresponding to the combination of the GSH sequence and the sequence SEQ ID NO: 11,

GSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 31, corresponding to the combination of the GSH sequence and the sequence SEQ ID NO: 12,

GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 32, corresponding to the combination of the GSH sequence and the sequence SEQ ID NO: 13,

GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D.

A subject of the invention is also the protein MabA corresponding to the sequence SEQ ID NO: 1, or the proteins derived from the protein MabA defined above, such as the derived proteins corresponding to the sequences SEQ ID NO: 3, 5, 7, 8, 9, 10, 11, 12, or 13, the first seven amino acids of which are deleted, namely the following sequences:

the following sequence SEQ ID NO: 33, corresponding to the sequence SEQ ID NO: 1 the first seven amino acids of which are deleted:

GAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 34, corresponding to the sequence SEQ ID NO: 3 the first seven amino acids of which are deleted:

GAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 35, corresponding to the sequence SEQ ID NO: 5 the first seven amino acids of which are deleted:

GAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 36, corresponding to the sequence SEQ ID NO: 7 the first seven amino acids of which are deleted:

GAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

the following sequence SEQ ID NO: 37, corresponding to the sequence SEQ ID NO: 9 the first seven amino acids of which are deleted:

GAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 38, corresponding to the sequence SEQ ID NO: 10 the first seven amino acids of which are deleted:

GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 39, corresponding to the sequence SEQ ID NO: 11 the first seven amino acids of which are deleted:

GAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 40, corresponding to the sequence SEQ ID NO: 12 the first seven amino acids of which are deleted:

GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D,

the following sequence SEQ ID NO: 41, corresponding to the sequence SEQ ID NO: 13 the first seven amino acids of which are deleted:

GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

in which X₁ represents D or E, and X₂ represents H or D.

The invention also relates to the protein MabA and the abovementioned derived proteins, characterized by their specific enzymatic activity of the substrates of the long-chain type β-ketoacyl, in particular between 8 and 20 carbon atoms, such as β-ketooctanoyl-CoA, or β-ketododecanoyl-CoA.

A more particular subject of the invention is the protein MabA and the abovementioned derived proteins, the main characteristics of the three-dimensional structure of which, at a resolution of 1.6-2.0 angströms, detected by X-ray diffraction analysis of the crystals of said proteins, are as represented in FIG. 1 for the recombinant protein MabA corresponding to the sequence SEQ ID NO: 15, in FIG. 2 for the derived protein MabA C(60)V corresponding to the sequence SEQ ID NO: 16, and in FIG. 3 for the derived protein MabA C(60)V/S(144)L corresponding to the sequence SEQ ID NO: 17.

The invention also relates to the protein MabA and the abovementioned derived proteins, in crystallized form.

The invention relates more particularly to the crystals of abovementioned proteins, as obtained by the hanging-drop vapour diffusion method, by mixing said proteins (1 μl of a 10 mg/ml solution) with a solution of polyethylene glycol, CsCl (150-300 mM), and glycerol (10%) in a buffer (PIPES) at pH 6.2.

A subject of the invention is also the crystals of abovementioned proteins, as obtained according to the crystallization method described above, said method being carried out from proteins purified using the abovementioned buffers more particularly used for obtaining proteins of the invention intended for crystallography studies.

The invention also relates to the abovementioned crystals of the recombinant protein MabA corresponding to the sequence SEQ ID NO: 15, the atomic coordinates of the three-dimensional structure of which are represented in FIG. 1, and having the following characteristics:

cell parameters:
a=81.403 angströms, b=116.801 angströms, c=52.324 angströms,
α=β=90.00°, γ=122.30°,

space group: C2,

maximum diffraction=2.05 angströms.

The invention also relates to the abovementioned crystals of the protein C(60)V corresponding to the sequence SEQ ID NO: 16, the atomic coordinates of the three-dimensional structure of which are represented in FIG. 2, and having the following characteristics:

cell parameters:
a=82.230 angströms, b=118.610 angströms, c=53.170 angströms,
α=β=90.00°, γ=122.74°,

space group: C2,

maximum diffraction=2.6 angströms.

A subject of the invention is also the abovementioned crystals of the protein C(60)V/S(144)L corresponding to the sequence SEQ ID NO: 18, the atomic coordinates of the three-dimensional structure of which are represented in FIG. 3, and having the following characteristics:

cell parameters:
a=81.072 angströms, b=117.022 angströms, c=53.170 angströms,
α=β=90.00°, γ=122.42°,

space group: C2,

maximum diffraction=1.75 angströms.

A more particular subject of the invention is the crystals of MabA and abovementioned derived proteins, in which said proteins are bound to a ligand, namely a molecule capable of binding to the protein MabA or to the proteins derived from the latter, more particularly at the level of their active site mainly delimited by the amino acids situated in positions 21 to 28, 45 to 48, 60 to 63, 87 to 100, 112, 138 to 157, 183 to 212, and 240 to 247 of the proteins corresponding to the sequences SEQ ID NO: 1, 3, 5, 7, 8, 9, 10, 11, or 13, or in positions 41 to 48, 65 to 68, 80 to 83, 107 to 120, 132, 158 to 177, 203 to 232, and 260 to 267, of the proteins corresponding to the sequences SEQ ID NO: 15 to 23, or in positions 24 to 31, 48 to 51, 63 to 66, 90 to 103, 115, 141 to 160, 186 to 215, and 243 to 250, of the proteins corresponding to the sequences SEQ ID NO: 24 to 32, or in positions 14 to 11, 38 to 41, 53 to 56, 80 to 93, 105, 131 to 150, 176 to 205, and 233 to 240, of the proteins corresponding to the sequences SEQ ID NO: 33 to 41, said crystals being as obtained by soaking or co-crystallization of the recombinant protein MabA in purified form, or of a recombinant protein derived from the abovementioned protein MabA, in the presence of said ligand, in particular under the crystallization conditions defined above.

The invention also relates to the nucleotide sequences coding for a protein derived from the protein MabA as defined above.

A more particular subject of the invention is therefore the nucleotide sequence coding for the derived protein C(60)V (SEQ ID NO: 3), and corresponding to the following sequence SEQ ID NO: 2:

atgactgccacagccactgaaggggccaaacccccattcgtatcccgttc
agtcctggttaccggaggaaaccgggggatcgggctggcgatcgcacagc
ggctggctgccgacggccacaaggtggccgtcacccaccgtggatccgga
gcgccaaaggggctgtttggcgtcgaagttgacgtcaccgacagcgacgc
cgtcgatcgcgccttcacggcggtagaagagcaccagggtccggtcgagg
tgctggtgtccaacgccggcctatccgcggacgcattcctcatgcggatg
accgaggaaaagttcgagaaggtcatcaacgccaacctcaccggggcgtt
ccgggtggctcaacgggcatcgcgcagcatgcagcgcaacaaaattcggt
cgaatgatattcataggttcggtctccggcagctggggcatcggcaacca
ggccaactacgcagcctccaaggccggagtgattggcatggcccgctcga
tcgcccgcgagctgtcgaaggcaaacgtgaccgcgaatgtggtggccccg
ggctacatcgacaccgatatgacccgcgcgctggatgagcggattcagca
gggggcgctgcaatttatcccagcgaagcgggtcggcacccccgccgagg
tcgccggggtggtcagcttcctggcttccgaggatgcgagctatatctcc
ggtgcggtcatcccggtcgacggcggcatgggtatgggccac

or any sequence derived by degeneration of the genetic code and coding for the protein C(60)V.

A subject of the invention is therefore also the nucleotide sequence coding for the derived protein S(144)L (SEQ ID NO: 5), and corresponding to the following sequence SEQ ID NO: 4:

atgactgccacagccactgaaggggccaaacccccattcgtatcccgttc
agtcctggttaccggaggaaaccgggggatcgggctggcgatcgcacagc
ggctggctgccgacggccacaaggtggccgtcacccaccgtggatccgga
gcgccaaaggggctgtttggcgtcgaatgtgacgtcaccgacagcgacgc
cgtcgatcgcgccttcacggcggtagaagagcaccagggtccggtcgagg
tgctggtgtccaacgccggcctatccgcggacgcattcctcatgcggatg
accgaggaaaagttcgagaaggtcatcaacgccaacctcaccggggcgtt
ccgggtggctcaacgggcatcgcgcagcatgcagcgcaacaaaattcggt
cgaatgatattcataggttcggtctccggcctctggggcatcggcaacca
ggccaactacgcagcctccaaggccggagtgattggcatggcccgctcga
tcgcccgcgagctgtcgaaggcaaacgtgaccgcgaatgtggtggccccg
ggctacatcgacaccgatatgacccgcgcgctggatgagcggattcagca
gggggcgctgcaatttatcccagcgaagcgggtcggcacccccgccgagg
tcgccggggtggtcagcttcctggcttccgaggatgcgagctatatctcc
ggtgcggtcatcccggtcgacggcggcatgggtatgggccac

or any sequence derived by degeneration of the genetic code and coding for the protein S(144)L.

A subject of the invention is therefore also the nucleotide sequence coding for the derived protein C(60)V/S(144)L (SEQ ID NO: 7), and corresponding to the following sequence SEQ ID NO: 6:

atgactgccacagccactgaaggggccaaacccccattcgtatcccgttc
agtcctggttaccggaggaaaccgggggatcgggctggcgatcgcacagc
ggctggctgccgacggccacaaggtggccgtcacccaccgtggatccgga
gcgccaaaggggctgtttggcgtcgaagttgacgtcaccgacagcgacgc
cgtcgatcgcgccttcacggcggtagaagagcaccagggtccggtcgagg
tgctggtgtccaacgccggcctatccgcggacgcattcctcatgcggatg
accgaggaaaagttcgagaaggtcatcaacgccaacctcaccggggcgtt
ccgggtggctcaacgggcatcgcgcagcatgcagcgcaacaaaattcggt
cgaatgatattcataggttcggtctccggcctctggggcatcggcaacca
ggccaactacgcagcctccaaggccggagtgattggcatggcccgctcga
tcgcccgcgagctgtcgaaggcaaacgtgaccgcgaatgtggtggccccg
ggctacatcgacaccgatatgacccgcgcgctggatgagcggattcagca
gggggcgctgcaatttatcccagcgaagcgggtcggcacccccgccgagg
tcgccggggtggtcagcttcctggcttccgaggatgcgagctatatctcc
ggtgcggtcatcccggtcgacggcggcatgggtatgggccac

or any sequence derived by degeneration of the genetic code and coding for the protein C(60)V/S(144)L.

The invention also relates to any recombinant nucleotide sequence comprising the nucleotide sequence coding for the protein MabA, or comprising a nucleotide sequence coding for a protein derived from the protein MabA, as defined above, in combination with the elements necessary for the transcription of this sequence, in particular with a transcription promoter and terminator.

A subject of the invention is also any vector, in particular plasmid, containing a nucleotide sequence as defined above.

The invention also relates to the host cells transformed by an abovementioned vector, said cells being chosen in particular from bacteria such as E. coli, or any other microorganism used for the production of proteins.

A subject of the invention is also a process for the preparation of the recombinant protein MabA in purified form, or of recombinant proteins derived from the protein MabA, as defined above, characterized in that it comprises the following stages:

transformation of cells using an abovementioned recombinant vector,

culture of the cells thus transformed, and recovery of said proteins produced by said cells,

purification of said proteins according to the purification process described above.

The invention also relates to the use of the recombinant protein MabA in purified form, or of recombinant proteins derived from the protein MabA as defined above, or of abovementioned crystals, for the implementation of methods for designing or screening ligands of the protein MabA, and more particularly molecules capable of binding specifically at the level of the active site of the protein MabA, or proteins similar in structure to the protein MabA, and inhibiting the enzymatic activity of the latter, these inhibitors being chosen in particular from:

the steroid derivatives,

the derivatives of the antituberculous antibiotic isoniazid (isonicotinic acid hydrazide), such as the derivatives of the isonicotinoyl-NAD(P) adduct,

the derivatives of N-acetyl cysteamine or other simplified types of derivatives of the coenzyme A, comprising a grafted fluorophore making it possible to use the fluorescence spectroscopy method, in particular time-resolved, for the detection of protein-ligand interactions,

the inhibiting derivatives of the protein hihA of Mycobacterium tuberculosis.

A more particular subject of the invention is the abovementioned use of the recombinant protein MabA in purified form, or recombinant proteins derived from the protein MabA as defined above, or abovementioned crystals, for the implementation of methods for designing or screening ligands of the protein MabA capable of being used in pharmaceutical compositions, in particular within the framework of the treatment of pathologies linked to mycobacterial infections, such as tuberculosis linked to infection by Mycobacterium tuberculosis, or by Mycobacterium africanium, or leprosy linked to infection by Mycobacterium leprae, or mycobacteriosis linked to infection by opportunist mycobacteria, such as Mycobacterium avium, Mycobacterium fortuitum, Mycobacterium kansasii, Mycobacterium chelonae.

The invention also relates to any method for screening ligands of the protein MabA, characterized in that it comprises the following stages:

being brought into the presence of the recombinant protein MabA in purified form, or a recombinant protein derived from the protein MabA as defined above,

detection of any bond between said protein and the ligand tested by measurement, after fluorescence excitation, in particular at 300 nm, of the intensity of fluorescence of said protein emitted between 300 and 400 nm (corresponding essentially to the emission of fluorescence of the single tryptophan W145), and comparison of the intensity of fluorescende emitted in a test in the absence of ligand, the binding of a ligand in the MabA active site being characterized by a quenching of fluorescence.

A subject of the invention is also any method for screening ligands inhibiting the protein MabA, characterized in that it comprises the following stages:

being brought into the presence of the recombinant protein MabA in purified form, or a recombinant protein derived from the protein MabA as defined above, in a reaction medium comprising a substrate, such as a β-ketoacyl derivative defined above, the coenzyme NADPH and the ligand tested,

detection of a potential inhibiting ability of the ligand tested, by measurement of the enzymatic activity of said protein by kinetic measurement of the absorbance, in particular at 340 nm, and comparison of the gradient of the optical density curve as a function of time with the gradient obtained in a test in the absence of ligand.

The invention also relates to any method for screening ligands of the protein MabA, characterized in that it comprises the following stages:

being brought into the presence of the recombinant protein MabA in purified form, or of a recombinant protein derived from the protein MabA as defined above, with the ligand tested,

analysis of the three-dimensional structure of the complex formed in soluble phase between said protein and said ligand, in particular by NMR, and by fluorescence.

A more particular subject of the invention is any method for screening ligands of the protein MabA, characterized in that it comprises the following stages:

co-crystallization of the ligand tested and the recombinant protein MabA in purified form, or of a recombinant protein derived from the protein MabA as defined above, in particular under the crystallization conditions defined above, in order to obtain the abovementioned crystals,

or soaking of the crystals of the protein MabA or of a derived protein as defined above, in optimized solutions containing potential ligands,

analysis of the three-dimensional structure of the abovementioned crystals, in particular by X-ray diffraction (with a view to selecting the ligands having an optimum ability to occupy and block the active site of said proteins).

The invention also relates to the use of the coordinates of the three-dimensional structure of the recombinant protein MabA in purified form, or a recombinant protein derived from the protein MabA as defined above, said coordinates being represented in FIGS. 1 to 3, if appropriate in combination with the coordinates of the active site of these proteins as defined above, for the implementation of methods for designing or screening ligands of the protein MabA (advantageously computer-aided).

A more particular subject of the invention is therefore any method for designing or screening ligands of the protein MabA, comprising the use of the coordinates of the three-dimensional structure of the recombinant protein MabA in purified form, or of a recombinant protein derived from the protein MabA as defined above, said coordinates being represented in FIGS. 1 to 3, for screening in silico the virtual combinatorial libraries of potential ligands, advantageously using appropriate computer software, and the detection and rational structural optimization of the molecules capable of binding to said protein.

A subject of the invention is also any method of rational design as defined above, carried out starting with known inhibitors of MabA or inhibitors of proteins homologous to MabA (of the same SDR or RED structural family, and exhibiting more than 10% identity with MabA throughout the peptide sequence), for which the fine three-dimensional structure of the complex between said inhibitor and the recombinant protein MabA in purified form, or a recombinant protein derived from the protein MabA, as defined above, was determined, and rational structural optimization of said inhibitors. We have shown that the activity of MabA was inhibited in vitro by an INH-NADP(H) adduct. This action mechanism of isoniazid (INH) on MabA is similar to the action mechanism of isoniazid on the protein InhA, target of the INH. Other proteins forming part of the RED superfamily have a three-dimensional structure comparable to that of MabA, including a steroid dehydrogenase (PDB1HSD), tropinone reductases (e.g. PDB1AE1), a trihydroxynaphthalene reductase (PDB1YBV) and a mannitol dehydrogenase (PDB1H5Q), and were co-crystallized with inhibitors.

The invention is further illustrated by means of the detailed description which follows of obtaining the recombinant protein MabA, and the proteins MabA C(60)V, S(144)L, and C(60)V/S(144)L, in purified form, their enzymatic properties, as well as crystals of these proteins and their atomic coordinates.

The atomic coordinates of the recombinant protein MabA (corresponding to SEQ ID NO: 15), and the proteins MabA C(60)V(corresponding to SEQ ID NO: 16), and C(60)V/S(144)L (corresponding to SEQ ID NO: 18), are respectively represented in FIGS. 1 to 3, which show from left to right the atomic number, name of the residues, chain number, x, y, z, coordinates, occupation, and factor B.

### EXPERIMENTAL PART

Tuberculosis, an infectious disease caused by Mycobacterium tuberculosis, remains the major cause of mortality world-wide due to a single infectious agent. According to the World Health Organization, 8 million cases of tuberculosis appear each year, resulting in 3 million deaths (Dolin et al., 1994). Whilst it has always posed a serious public health problem in developing countries, tuberculosis is reappearing in the developed countries. The precarious conditions of certain social groups and the deterioration in health systems, consequences of the world economic crisis, have promoted this recrudescence of tuberculosis. Similarly, the endemic of infection by the human immunodeficiency virus (HIV) and the appearance of strains of M. tuberculosis resistant to one or more antibiotics have also strongly contributed to this phenomenon (Barnes et al., 1991). The emergence of multi-resistant tuberculosis, defined as resistance to the two antibiotics which form the basis of antituberculous treatment, isoniazid (Rimifon, INH) and rifampicin (RMP), is a threat to the control of tuberculosis. Patients infected by the strains resistant to several antibiotics are extremely difficult to care for and the necessary treatment is toxic and expensive. Up to 30% of all of the resistant clinical isolates of M. tuberculosis are resistant to isoniazid (Cohn et al., 1997). It is therefore important to better understand the mechanisms of resistance to isoniazid established by the mycobacteria in order to be able, on the one hand, to develop rapid techniques allowing the detection of resistances and, on the other hand, to develop new anti-mycobacterial agents which are effective against the resistant strains.

Starting from numerous works carried out on isoniazid, it has been possible to identify a direct target of this antibiotic: a metabolism specific to mycobacteria, the biosynthesis of mycolic acids [(Winder & Collins, 1970); (Takayama et al., 1972); (Quémard et al., 1991)]. These very long chain fatty acids are major and characteristic constituents of the mycobacterial envelope. Thanks to the use of the tools of molecular biology in M. tuberculosis, a molecular target of isoniazid, the protein InhA, probably involved in the biosynthesis route of mycolic acids has been characterized [(Banejee et al., 1994); (Quemard et al., 1995)]. InhA belongs to an enzymatic system responsible for the elongation of the fatty acids (Marrakchi et. al., 2000). This system containing the protein InhA, a target of isoniazid, participates in the biosynthesis of mycolic acids and therefore represents an enzymatic complex the components of which are interesting to study, as potential targets of new antituberculous antibiotics. We have therefore studied the biochemical properties of MabA, one of the proteins of the complex containing InhA. The molecular modelling of the three-dimensional structure of this protein, which catalyzes the reduction of β-ketoacyl derivatives, has shown that MabA and InhA form part of the same structural family. The study of the effect of isoniazid on the enzymatic activity of MabA suggests that the antibiotic inhibits the protein by a mechanism similar to the action on InhA. Thus, MabA represents a useful target for the design of inhibitors of the biosynthesis of fatty acids in mycobacteria.

Within the framework of the work on the present invention, the mabA gene of M. tuberculosis was cloned in E. coli, in an expression vector. The protein is produced in a large quantity by this recombinant strain, as a fusion protein possessing an N-terminal poly-histidine tag. Purification of the protein is carried out in a single stage by column chromatography, producing several mg of purified protein. A wild-type MabA monomer possesses 247 amino acids and has a size of 25.7 kDa; the fusion monomer is 27.7 kDa. Several experimental methods (analytical ultracentrifugation, gel permeation, light diffusion, crystallography etc.) made it possible for us to show that the native protein was mainly tetrameric, originating from the self-association of two dimers. Physico-chemical properties (stability in different buffer media, at different temperatures, fluorescence emission spectra etc.) and the main enzymatic properties of MabA (Kd, Km and kcat for the coenzyme and -β-ketoacyl-CoA- substrates of different chain lengths) were determined. The purified recombinant protein is functional; this is a β-ketoacyl reductase, NADPH-dependent, and specific to long-chain substrates (C12-C20). We have shown that MabA formed part of the elongation system of mycobacterial fatty acid, FAS-II, and catalyzes the 2nd stage of the elongation cycle.

The three-dimensional structure of the protein MabA was resolved by crystallography to 2.05 Å resolution after development of the conditions for cryostabilization of the crystals. MabA forms part of the structural super-family of the SDR (Short-Chain Reductases) or RED (Reductases, Epimerases, Dehydrogenases) proteins. It is homologous to the KARs (ketoacyl-ACP reductases), but represents a particular member of this family, by of the structure of the substrate-binding pocket. The latter has a more hydrophobic character than that of the homologous proteins. The presence of the single tryptophan residue in the substrate-binding pocket allowed us to carry out fluorescence spectroscopy experiments, which demonstrated a more marked affinity of MabA for long-chain substrates (C8-C20) compared with the C4 substrate. These results, which correlate with the enzymatic kinetic data, demonstrate a structure-function relation between the hydrophobicity of the binding site of the substrate and the affinity of MabA for unusually long substrates in the bacteria.

The distinct properties of MabA relative to the other homologous proteins make it a target of choice for the design of potential antibiotics. This design will use several parallel approaches:

1. rational design starting with known inhibitors of MabA or homologous proteins

2. high-throughput screening of virtual combinatorial libraries

3. high-throughput screening of real combinatorial libraries.

We have shown that the activity of MabA was inhibited in vitro by an INH-NADP(H) adduct. This action mechanism of isoniazid (INH) on MabA is similar to the action mechanism of isoniazid on the protein InhA, target of NH. Other proteins forming part of the RED super-family have a three-dimensional structure comparable to that of MabA, including a steroid dehydrogenase (PDB1HSD), tropinone reductases (e.g.: PDB1AE1), a trihydroxynaphthalene reductase (PDB1YBV) and a mannitol dehydrogenase (PDB1H5Q). Thus, approach (1) is based on the use of the structure of ligands (e.g. isoniazid derivatives, steroids) of these different proteins for the design of other potential inhibitors of MabA, of derived structures. Rational design of course involves the use of the crystalline structure of MabA and of the computer-aided molecular docking method. Similarly, if approaches (2) and (3) provide new types of potential ligands, the latter will be able to form the basis of new rational designs.

The invention therefore provides a conceptual approach for the development of inhibitors of the activity of the protein MabA. It also offers a method of experimental validation, on the one hand, of the specific binding of these molecules to the active site of MabA (fluorescence spectroscopy) and on the other hand, of the inhibiting ability of these molecules by a simple enzymatic test (enzymatic kinetics by monitoring by spectrophotometry).

I) Study of the Protein MABA

We have shown that the FAS-II elongation system contains the protein InhA, a target of isoniazid. Moreover, the fact that this system is probably involved in the biosynthesis of mycdlic acids, compounds specific to mycobacteria, makes FAS-II a target of choice for anti-mycobacterial agents. Study of the enzymes which make up this system is therefore a useful approach for research into new targets of antibiotics.

The strong inhibition of the activity of FAS-II by isoniazid, and above all the fact that no biosynthesis intermediate, and in particular the substrates of InhA, accumulate under the effect of the INH suggest that another target of the antibiotic could exist in addition to InhA. The protein MabA, coded by a gene contiguous to inhA on the chromosome of M. tuberculosis, probably forms part of the same enzymatic system as InhA. Several data suggest that MabA could be a target of isoniazid. On the one hand, point mutations in the promoter region of the mabA-inhA locus of clinical isolates of M. tuberculosis lead to the overproduction of the proteins downstream and correlate with a phenotype of resistance to INH. This suggests that in addition to the overproduction of InhA, induced by these mutations, the overproduction of MabA could also participate in the resistance, if this protein interacts with isoniazid. On the other hand, study of the effect of isoniazid (2 mM) on the purified enzymes of the isolated FES system of M. avium has shown that two stages of the system are sensitive to INH, β-ketoacyl reductase (93% inhibition and Ki=353 μM), which is the most sensitive, and enoyl-reductase (26% inhibition and Ki=5.5 mM) (Kikuchi et al., 1989).

We therefore decided to purify the protein MabA and to study certain of its biochemical properties, by adopting a strategy of overproduction of the protein in a prokaryotic system.

1. Overproduction of MABA in Escherichia Coli

Carrying out an enzymatic study and producing anti-MabA antibodies for the intracellular location of MabA, required the obtaining of the pure protein, in soluble form and in a large quantity. In order to overproduce MabA, we used a system of expression and purification in Escherichia coli, which is simple and very effective for the overexpression of prokaryotic genes. The development of an experimental procedure in order to achieve sufficient overproduction, whilst obtaining the protein in soluble form, required optimization at several levels of the overexpression and purification diagram.

1.1 Cloning of the mabA Gene of Mycobacterium Tuberculosis H37Rv

The determination of the complete sequence of the genome of Mycobacterium tuberculosis H37Rv (Cole et al., 1998) and the development of the techniques of molecular biology allowing the manipulation of recombinant DNA, facilitated the production and study of the protein of interest, MabA.

1.1.1 Description of the Expression System of mabA in Escherichia Coli

Choice of the Expression Vector pET (Plasmid for Expression by T7 RNA Polymerase)

In the expression vector used, pET-15b (Novagen), the target gene is cloned under the control of the transcription and translation signals of the bacteriophage T7. The mabA (fabG1) gene of 741 base pairs, coding for the protein MabA was amplified by polymerase chain reaction (PCR) from the cosmid MTCY277 (Institut Pasteur), and cloned between the restriction sites NdeI and Xho of the plasmid. This plasmid offers the advantage of being able to obtain in NH2-terminal fusion of the recombinant protein, a poly-histidine sequence, cleavable, allowing a rapid purification of the protein by affinity chromatography. The construction therefore comprises upstream of the mabA gene, a sequence coding for 6 successive histidines and the site of cleavage by thrombin.

Choice of the Host Strain

The host strain of E. coli chosen, BL21(λDE3) (Novagen), has the advantage of having the 2 inactive ompT and lon genes. The ompT [(Studier & Moffatt, 1986); (Studier et al., 1990)] and lon genes (Phillips et al., 1984) code respectively for the parietal protease (responsible for the degradation of heterologous proteins) and the main cytoplasmic protease (responsible for the degradation of poorly folded or unstable proteins). BL21(λDE3) is lysogen for the bacteriophage DE3 (λ derivative), and therefore carries a chromosomal copy of the gene of the T7 RNA polymerase under the control of the lacUV5 promoter, which is IPTG (isopropyl-β-D-thiogalactopyranoside) inducible. The addition of IPTG to a culture of the lysogen induces the expression of T7 RNA polymerase, which in turn will transcribe the target DNA on the plasmid.

1.1.2 Transformation and Selection

The transformation of the competent E. coli strain BL21(λDE3) by the pET-15b::mabA plasmid was carried out by thermal shock (Material and Methods). The effectiveness of transformation obtained is 5.9 103 CFU/μg of DNA. The weak effectiveness of transformation characterizing the strains of B coli from which BL21(λDE3) is derived is noted.

The selection of the cells having incorporated the plasmid is carried out thanks to the acquisition of resistance to ampicillin. In our selection experiments, we preferred to use carbenicillin, a stable β-lactain, rather than ampicillin which is known to be rapidly degraded by the β-lactainases secreted by the resistant bacteria.

1.1.3 Verification of the Sequence of the Cloned mabA Gene

In order to verify that no mutation was introduced during the PCR amplification stage of mabA, the sequence of the cloned gene was analyzed. No mutation was found; the cloned sequence is identical to that carried by the original cosmid.

1.2. Heterologous Expression and Optimization

The optimization of the expression of a heterologous gene requires a preliminary small-scale study in order to determine the choice of culture conditions and induction parameters (OD, temperature, concentration of the inducer and induction time). The development of these conditions allowed us to define the procedure to be followed in order to obtain a sufficient overproduction of the protein which is visible in SDS-PAGE. However, despite our efforts to reproduce the overexpression on a larger scale, we did not succeed in producing the protein MabA by the bacteria induced. The most plausible hypothesis was the loss of the plasmid, despite the maintenance of the cultures in medium containing the antibiotic. Faced with this problem, the plasmid stability test in a dish (Material and Methods) offered us a rapid and reliable means of verifying, in the cultures before induction, the presence of the target plasmid on the one hand, and the ability of the bacteria transformed, in culture, to express the heterologous DNA, on the other hand.

The heterologous expression of mabA in E. coli proved particularly sensitive to the culture conditions which affect the stability of the plasmid. For optimal expression, it is important to fulfill two conditions:

The transforming colonies must be fresh (coming directly from a transformation or a plating by stria from a liquid stock stored at −70° C).

The number of generations between the transformation of the bacteria by the plasmid and the induction of the expression must be reduced to a minimum (avoiding intermediate cultures).

2. Purification of MabA

2.1 Solubility of the Overproduced Protein and Optimization

In order to establish the purification strategy, it is important to know whether the protein is produced in soluble form, or localized in the inclusion body (aggregates of proteins).

The small-scale tests to determine the solubility under optimum induction conditions revealed certain interesting and unexpected points. The first is that the variations applied to the induction parameters (temperature, OD or duration of induction), aimed at improving the solubility, do not seem to have significant consequences on the preferential production of the protein MabA in such or such a form. On the other hand, we were surprised to note that the technique adopted for lysing the bacteria modulated the distribution of the protein between the soluble and insoluble fractions. For example, cold sonication in a reduced volume (concentration factor of the culture CF>20) is probably responsible for the precipitation of MabA in the pellet (insoluble fraction). The effect of the high local temperatures engendered by the ultrasonics could explain this phenomenon of aggregation-precipitation of MabA. The lysis of a bacterial suspension at a lower cell density makes it possible to avoid the precipitation of MabA during sonication.

A study carried out on the effects of ultrasonics on enzymes (Coakley et al., 1973) reveals that the cellular extracts prepared by ultrasonic disintegration are sensitive to the damage caused by the free radicals, which are-probably generated by the ultrasonics, as well as by the effect of high local temperatures. These authors show that the “damaging” effect during the lysis of the bacteria can be minimized by sonication at a high concentration in cells and in the presence of components of the medium such as the sugars (acting as “scavengers” of radicals). These conclusions do not seem to be in agreement with our results which show that at a lower cell density during the lysis of MabA, the protein is found in the “soluble” fraction. However no theory can be advanced as to the activity of the protein under these conditions.

After comparison of several lysis techniques on the desired production scale, we opted for a lysis by lysozyme, followed by a freeze-thaw cycle. According to this protocol, and under the induction conditions adopted [OD600=0.8, 2 hours at 37° C., 0.8 mM IPTG], a large part of the protein MabA is found in the soluble fraction.

2.2 Purification System

The obtaining of the protein MabA with a poly-His (H-MabA) tag facilitates its purification. In fact, the high affinity of the histidine residues for the metal ions makes it possible to use the imobilized metal ion affinity chromatography (IMAC) method. One of the matrices most used for its effectiveness is nickel-nitrilotriacetate Ni-NTA-agarose (Qiagen). The NTA group has 4 chelation sites interacting with 4 of the 6 coordination sites of the metal ion Ni. The imidazole nuclei of the histidine residues bind to the nickel ions on the Ni-NTA matrix. The addition of imidazole molecules makes it possible, by competition with the histidine residues, to break the bonds between the proteins and the matrix, and to elute the bound poly-His protein. The affinity of a protein for the Ni-NTA-agarose matrix is a function of the number of histidine residues which it possesses and which are exposed to the matrix. Thus, by adjusting the imidazole concentration, different species of proteins having different degrees of affinity can be eluted. The very high affinity of the proteins having a poly-His tag for nickel makes it possible to separate it from the majority of the proteins co-produced by E. coli. Thus, if the binding of the protein H-MabA proves sufficiently specific, purification will be limited to the single stage of affinity chromatography.

2.3 Purification of MabA in Native Conditions

The development of the conditions for elution of the protein MabA on Ni-NTA-agarose resin is carried out on a small scale (50 μl), using the so-called resin sedimentation method in batches. Thanks to this technique, we were able to determine the different imidazole concentrations necessary for the elution of the protein MabA and elimination of the other proteins.

The purification adapted on a larger scale is carried out in open column with 500 μl of resin in suspension (Material and Methods). Moving from batch purification to column purification required an additional stage of development.

The protein fractions corresponding to the different purification stages are analyzed by SDS-PAGE. In the clarified lysate, the majority band obtained between 30 and 43 kDa and corresponding to MabA, provides evidence of a fairly large overproduction of the protein in soluble form, more than 50% of the soluble proteins of E. coli. After the stage of binding of the proteins on the resin, the fraction containing the non-bound proteins on the column is devoid of MabA, indicating an effective binding of the protein H-MabA to the Ni-NTA matrix. The elimination of other proteins weakly bound to the matrix (by the presence of histidines dispersed in their sequence), is obtained after extensive washings with 50 mM imidazole. An imidazole concentration equal to 175 mM is required in order to elute the protein H-MabA alone and in a very large quantity. The apparent masse of H-MabA deduced from its electrophoretic migration is estimated at 35 kDa.

2.4 Problems of Precipitation of MabA During the Purification

During the purification, we noted that the protein MabA, eluted at a very high concentration, immediately precipitated in the tube. This behaviour often observed for the proteins with a poly-His tag, is probably due to non-specific protein-protein interactions due to the very strong local protein concentration during the purification (TALONTM Metal affinity Resin—User Manual CLONTECH).

Attempts at solubilization of the protein eluted with detergents (Triton X-100, NP-40) proved to be in vain. It was therefore necessary to intervene before the elution of the protein. In order to prevent the precipitation of the protein, we carried out a treatment before and after purification. In order to verify whether the protein precipitates, the fraction which contains MabA after elution is centrifuged (5 minutes, at 12,000 g) then the supernatant and the pellet are analyzed by SDS-PAGE (Material and Methods). The pre-purification treatment consists of adding mild “solubilizing” agents to the lysate. After purification, the eluted protein is recovered directly in glycerol (50% final) (glycerol is a protective agent, much used for preserving the activity of the enzymes).

Three conditions were tested:

10% (v/v) glycerol alone

10% (v/v) glycerol+0.1% (v/v) Triton X-100 (non-ionic detergent)

10% (v/v) glycerol+0.05% (v/v) (7 mM) β-mercaptoethanol (reducing agent).

The three processes made it possible to improve the solubility of the protein MabA. It was noted however, during the use of 7 mM β-mercaptoethanol, that H-MabA begins to be eluted at a much lower imidazole concentration (50 M instead of 175 mM).

The solubilization of MabA in the presence of the three agents tested being comparable, we opted for the addition of 10% glycerol alone to the lysate.

2.5 Protocol Optimized for the Overproduction and Purification of H-MabA

The optimization of the conditions for overexpression and purification of H-MabA allowed us to adopt the following protocol:

200 ml of E. coli/h-mabA on LB+CBC 50 μg/ml are cultured up to OD600=0.8;

Expression is induced with 0.8 mM IPTG for 2 hours, at 37° C.;

The bacteria are sedimented by centrifugation for 15 minutes at 10,000 g, at 4° C. The pellet is taken up in 9 ml of lysis buffer (5 mM imidazole and 500 mM NaCl);

Lysozyme (0.5 mg/ml) and the protease inhibitors (0.113 mg/ml) are added;

Freezing is carried out overnight at −70° C;

Thawing is carried out for 1 hour at ambient temperature and treated by the DnaseI (5 μg/ml) and the RnaseA (10 μg/ml) in the presence of MgCl2 (10 mM), 15 minutes at 4° C.;

The lysate is centrifuged at 3,000 g then at 10,000 g, and the soluble fraction recovered;

The supernatant is centrifuged for 45 min at 44,000 g, at 4° C. and the “clarified lysate” recovered;

10% of pure glycerol (v/v) is added to the soluble fraction and deposited on a mini-column (500 μl of Ni-NTA-agarose phase). Incubation is carried out for 1 hour at 4° C. under gentle stirring;

The phase is washed with 5×4 ml of elution buffer with 5 mM imidazole;

Pre-elution is carried out with 8×500 μl of 50 mM imidazole;

Elution is carried out with 8×500 μl of 175 mM imidazole;

Washing is carried out with 10×500 μl of250 mM imidazole;

The fractions containing the protein are collected according to their concentration and their purety. The protein is collected directly in an equal volume of pure glycerol, followed by dialysis against 50 mM potassium phosphate buffer, pH 7.2, containing 50% glycerol and stored at −20° C.

Elution buffer: 50 mM potassium phosphate buffer, pH 7.8

2.6 Purification Yield

Thanks to the expression and purification system used, it was possible to purify the protein H-MabA to homogeneity in a single stage. In order to know approximately the concentration of the protein solution, its ultraviolet absorbance at 280 nm was determined. Knowing the absorbance of the tyrosine and tryptophan residues of the protein (Material and Methods), the theoretical molar extinction coefficient of MabA was deduced (ε_{280 nm}=9530 M⁻¹cm⁻¹) and the molar concentration of the purified solution was estimated at 40 μM.

The best purification yield (percentage of purified protein relative to all of the total proteins deposited on the column) obtained is 57%. Starting with 200 ml of culture, we obtained approximately 20 mg of pure protein MabA (yield 100 mg/l of culture), which is very satisfactory.

3. Characterization of the Purified Protein MabA

3.1. Verification of the Peptide Sequence

The mabA gene cloned in pET-15b was sequenced, no mutation was found. The primary sequence of the wild-type protein MabA has 247 amino acids. The poly-histidine tag of the recombinant protein adds 19 amino acids to it (266 amino acids in total). The sequencing of the first 20 amino acids of the overexpressed protein MabA was carried out (Biomerieux, Lyon). We were able to verify the identity of the protein on the amino-terminal part and detect the loss of the first methionine of the poly-His tag. The elimination of the amino-terminal methionine from proteins by post-translational proteolysis is very frequent in E. coli.

3.2. Control of the Purity of the Sample

Analysis by denaturing electrophoresis (SDS-PAGE) and Coomassie blue staining of the eluate MabA shows a single band, indicating the homogeneity of the preparation. The purity of the protein was also verified by SDS-PAGE after staining with silver nitrate. No contaminant protein band is detected by this very sensitive development technique.

In order to determine the mass of the purified protein with precision, analysis by electrospray ionization mass spectrometry (ESI-MS) was carried out.

3;3 Determination of the Molecular Mass

Mass spectrometry makes it possible to verify very rapidly that the protein expressed has the expected mass. We analyzed a sample purified by electrospray ionization/mass spectrometry (ESI/MS), in collaboration with B. Monsarrat (IPBS, Toulouse). On the type of instrument used, the molecular mass of a protein is determined with a precision of 0.01% ( 1/10,000). The mass of the protein MabA predicted from the gene sequence (taking account of the poly-His tag) is 27,860 Da. Analysis by ESI/MS in direct introduction reveals a majority mass of 27,728±2 Da.

The difference between the theoretical mass and the measured mass (131 mass units) corresponds to the loss of the first methionine at the amino-terminal end, detected by the N-terminal sequencing of the protein. The molecular mass of the purified protein H-MabA thus determined is 27,728 Da.

The migration of H-MabA in denaturing electrophoresis towards 35,000 Da could be linked to the physico-chemical characteristics of the protein and/or to its native form.

3.4 Determination of the Quaternary Structure of MabA by Gel Filtration

Exclusion chromatography makes it possible to determiner the native form (quatemary structure) of the protein in solution at a given concentration and under the defined conditions of pH and ionic strength. Thanks to this technique, it is possible to establish a relation between the elution volume of the protein and its molecular weight, via a calibration curve. The calibration curve is deduced from the elution profiles of the standard proteins (Pharmacia).

The elution of the protein MabA (0.66 mg) was carried out under the same conditions as those of the standard proteins. On the chromatogram, an eluted asymmetrical peak is observed towards the high molecular weights. The elution volume corresponding to the top of the peak indicates that the majority molecular mass (94.6 kDa) is comprised between 110,916 Da and 83,187 Da, corresponding to the tetrameric or trimeric form of H-MabA, respectively. The slight shoulder distinguished on the profile (around 57.7 kDa) shows the presence, in a smaller proportion, of a dimeric form (55,458 Da) of the protein. These results suggest that there is probably a dimer-tetramer equilibrium of the protein MabA. Study of the three-dimensional structure by molecular modelling of MabA favours this hypothesis (see hereafter). It is however important to stress that the determination of the oligomeric structure by gel filtration is dependent on the tested conditions and in particular the concentration of the protein solution. The possibility of the presence of the protein MabA in vivo in the tetrameric form will be discussed hereafter.

3.5 A Few Physico-Chemical Properties of MabA

Certain physico-chemical properties of MabA can be deduced from its peptide sequence using one of the calculation programmes available on the Internet (aBi). The sequence of 266 amino acids of the protein H-MabA produced gives a calculated mass equal to 27729.37 Da. It corresponds to that determined by ESI/MS, to approximately 1 mass unit. The other characteristics are summarized in Table I hereafter.

TABLE I
Physico-chemical characteristics of H-MabA

| Parameter | Value | Note |
|---|---|---|
| Sequence | 266 amino acids | Absence of the N- |
| | | terminal methionine |
| Molecular mass | 27,729 Da | Verified by ESI/MS |
| Molar extinction* | 9530 M⁻¹ cm⁻¹ | Low, presence of a |
| coefficient ε _{280 nm} | | tryptophan and three |
| | | tyrosines in the |
| | | sequence |
| Absorbance* at 280 nm | A_{280 nm}^{0.1%} = 0.348 | |
| of a 0.1% solution | | |
| (1 mg/ml) | | |
| Isoelectric point Ip* | 9.79 | net charge (+) at |
| | | neutral pH |

*estimated by calculation, from the peptide sequence.

4. Catalytic Activity of MabA

The attribution of a potential activity to a protein of unknown function is often based on the similarity of sequence which it has with known proteins. Examination of the primary structure of the protein MabA demonstrates a strong identity with the sequence of the β-ketoacyl-ACP reductase FabG of E. coli (44% identity over 241AA), as well as with the β-ketoacyl-ACP reductases of other bacteria or plants. This enzymatic activity corresponds to one of the stages of the classic biosynthesis route of fatty acids. The elongation of fatty acids by the mycobacterial system FAS-II involves the protein InhA, which catalyses the NADH-dependent enoyl-ACP reduction stage. The elongation system FAS-II being comprised of several aggregated enzymes, it was logical to envisage the presence of the protein MabA combined with InhA in the same enzymatic complex. A strong argument in favour of the involvement of MabA and InhA in the same metabolic route rests on the operon organization of the mabA and inhA genes in M. tuberculosis. The genes involved in the biosynthesis of fatty acids are often grouped into “clusters” as for example in E. coli (Rawlings & Cronan, 1992) and in Vibrio harveyi (Shen & Byers, 1996).

Detecting the β-ketoacyl reductase activity of the purified protein MabA is the first stage of its characterization as potential partner of InhA in the biosynthesis of fatty acids.

4.1 Enzymatic Characterization of the Protein MabA

4.1.1. Demonstration of the Catalytic Activity of MabA

4.11.1. Description of the Enzymatic Test

The activity of the purified-protein H-MabA was first tested in the presence of the only commercial β-ketoacyl-CoA, acetoacetyl-CoA, and NADPH as electron donor. The addition of pure MabA to the substrates triggers the reaction. The evolution of the reaction is monitored for 5 minutes by measuring the reduction in absorbance at 340 nm, expressing the disappearance of the NADPH co-substrate in favour of its oxidized form NADP⁺ (which does not absorb at this wavelength).

Under the standard enzymatic test conditions defined (see Material and Methods), H-MabA is capable of reducing acetoacetyl-CoA. The purified protein H-MabA is therefore functional: it corresponds to a β-ketoacyl reductase (KAR: keto-acyl reductase). The presence of the poly-His tag in N-terminal position does not seem to impede its activity.

The substitution of NADPH by NADH at the same concentration in the kinetics test leads to a total loss of the activity. The protein MabA is therefore strictly NADPH-dependent. The presence in the peptide sequence of MabA of an NADP(H) binding unit confirms this result. The KARs of other organisms are most often NADPH-dependent and have a strict specificity for the nucleotide coenzyme.

4.1.1.2 Parameters Affecting the Activity of the Protein MabA

The activity of an enzyme is directly affected by the concentration of its substrates, but also by parameters such as the nature of the buffer, pH, the ionic strength, temperature. In order to optimize the reaction conditions, we studied the effect of the pH and ionic strength on the activity of MabA.

Effect of the pH

We evaluated the effect of the pH on the enzymatic activity of MabA using sodium phosphate buffer solutions with a pH of 5.0 to 8.0 in the reaction medium. Comparison of the results for the chosen pH range shows that the optimum activity of MabA is obtained for a pH equal to 5.5. However, at an acid pH (5.0 to 6.5), the NADPH is very unstable and is oxidized spontaneously, which leads to a variation in absorbance over time in the absence of enzyme. We therefore decided to work at pHs close to physiological pH (between 7.0 and 7.5), for which the base line has a negligible gradient compared with the catalysis gradient (less than 5-10%). Other β-ketoacyl-ACP reductases have an acid optimum pH (around 6.0-6.5) [(Shimnakata & Stumpf, 1982); (Caughey & Kekwick, 1982)].

If MabA has a better activity at pH 5.5, this is probably linked to a protonation event involved in the binding of the substrates or in the catalysis. This event could concern two His residues of the protein, H46 and H247 (the pKa of the imidazole nucleus of the histidine residue is equal to 6.0-6.5), potentially involved in the active site, according to the structural model of MabA.

Effect of the Ionic Strength

The MabA activity tested is constant for phosphate buffer concentrations varying between 20 and 100 nM. We opted for an 80 mM buffer, pH 7.0.

Effect of Dilution

The enzymatic tests requiring a preincubation of H-MabA over time revealed that the catalytic activity decreases rapidly if the enzyme is incubated at a low concentration (molar concentration <1 μM). The inactivation by dilution of the β-ketoacyl-ACP reductases of E. coli and of plants (Brassica napus, Persea americana) has already been reported (Schulz & Wakil, 1971); (Sheldon et al., 1990); (Sheldon et al., 1992)].

4.1.2. Determination of the Kinetic Parameters of MabA

The characterization of an enzyme generally comprises the determination of the maximum reaction velocity, Vmax and of the “Michaelis constant”, Km, for each substrate. Knowledge of these parameters proves very useful for biochemical studies (comparison of the affinity for different substrates, interaction with other molecules, comparison of isoenzymes of different organisms) and in particular for defining the effectiveness of inhibitors or activators of the enzyme.

4.1.2.1 Measurement of the Km for NADPH

Determination of the kinetic parameters Vmax and Km begins with the estimation of the Km value, by testing two concentrations of substrate, one low and the other high. The initial reaction velocities are then determined for a preferably wide range of concentrations in substrate, if possible covering from Km/2 to 5 Km. We plotted the straight line S/v=f (S) or 1/v=f(1/S) in order to visualize the data, then we compared the Km and Vmax values calculated by this method and those obtained by the least error squares method. The values obtained are the average of three manipulations. The determination S/v=f (S) produces results close to those obtained by the least error squares method. The value of Km obtained for NADPH, 39 μM, is approximately five times greater than that of the protein InhA for its cofactor NADH (8 μM). This higher Km probably reflects a lesser affinity of MabA for its coenzyme. The Km's of the β-ketoacyl reductases of other organisms for their cofactor are of the same order of magnitude as that obtained for MabA.

4.1.2.2 Measurement of the Km for Acetoacetyl-CoA

The Km for the acetoacetyl-CoA, determined in the presence of NADPH, is 1582 μM. This relatively high Km is much greater than the Km described for other β-ketoacyl-ACP reductases of plants. The fact that MabA has a higher Km than these enzymes which belong to of synthesis systems de novo, therefore specific to short chain substrates would suggest that MabA could have a preference for substrates longer than 4 carbons. Study of the specificity of MabA for substrates with a longer hydrocarbon chain thus seemed to us doubly important, on the one hand in order to better characterize the enzymatic activity of this protein and on the other hand in order to compare the substrate-specificity of MabA and that of InhA. The protein InhA was shown to be specific to long chain substrates (12-24 carbon atoms), exhibiting no activity in the presence of the substrate with 4 carbons (crotonoyl-CoA), even at 8 mM (Quemard et al., 1995).

4.1.3. Determination of the Kinetic Constants for the Long Chain Substrates

The use of long chain substrates (C8 to C20) imposes constraints linked to their critical micellar concentration (CMC). The long chain acyl-CoAs are amphiphilic compounds and only form true solutions at allow concentration. Beyond the CMC, some of the molecules form micelles and the monomer concentration is fixed at the CMC, therefore different from the total concentration. It was therefore important to use solutions with concentrations below the CMC. In a study of the physical properties of acyl-CoAs (Constantinides & Steim, 1985), the CMC's of aqueous solutions of palitoyl-CoA (C16-CoA) and stearoyl-CoA (C18-CoA) determined are respectively 70 and 12 μM. The presence of an unsaturation (in position 9) in the case of oleyl-CoA (C18:1-CoA) raises its CMC to 33 M. The presence of a ketone function on the chain would in theory have a similar effect relative to the CMC. Using these data, we attempted to prepare solutions of β-ketothioester the concentration of which was above the CMC. The stock solutions used for the kinetics tests are 400 μM and 100 μM for the C8 and C12 β-ketothioesters, respectively.

4.1.3.1. Measurement of the Km for the C8 and C12 β-Ketoacyl-CoAs

We measured the kinetic parameters of MabA for β-ketooctanoyl-CoA (C8) and β-ketododecanoyl-CoA (C12). The protein has a Km (60 μM) for the C8 substrate 25 times lower than that of the C4 (1582 μM). The C12 derivative also proves a much better substrate (Km of 9 μM). There also, the values obtained by the Hanes method and that of the “least error squares” method are similar. We calculated the Km/Vrmax ratio which reflects the affinity of the enzyme for its substrates. Kn/Vmax becomes lower as the substrate chain length increases. This correlation is due not only to the lower Km values, but also to higher Vmax values for the longer chains.

The kinetic constants Km and Vmax for C16 and C20 were determined. For those β-ketoacyl-CoAs with more than 12 carbon atoms, problems of inhibition by the substrate were encountered, also described in the case of the use of substrates of InhA of a size greater than C16. We therefore compared the initial reaction velocities at the same concentration (2 μM), in the presence of different β-ketoacyl-CoAs (C4 to C20). In order to measure the activity, it was necessary to use solutions of enzymes at different concentrations for the various β-ketothioester substrates. The protein MabA has a considerable preference for the 12-carbon substrate compared with the short substrates, and the C16 and C20 β-ketothioesters prove to be substrates at least as good as the C8. The reduction in the reaction velocity observed for the long chain of β-ketoesters could be linked to their low solubility (in the case where the real concentration of free molecules would be less than 2 μM).

4.1.3.2. Substrate Specificity and Involvement in an Elongation Route?

Although it has an activity in the presence of 4-carbon β-ketoacyl, the protein MabA nevertheless shows a clear preference for the C12-C16 substrates. The affinity of MabA for the long chain hydrocarbon substrates is compatible with the size and hydrophobic nature of the substrate-binding pocket. The protein InhA itself has a slightly different affinity, with a preference for longer C16-C24 substrates (Quémard et al., 1995). The enzymatic properties of MabA and InhA, in particular their specificity for medium to long chain substrates, goes in the direction of their belonging to the same fatty acids elongation system, FAS-II, which is itself specific to C12-C18 substrates.

The specificity of InhA substrate differs from that of the enoyl reductases of the type II systems of Spinacea oleracea (Shimakata & Stumpf, 1982)) or of E. coli (Weeks & Wakil, 1968), which have a preference for C6 and C8 substrates. Moreover, the β-hydroxyacyl dehydratase of the type II system of E. coli (Birge & Vagelos, 1972) is specific to short-chain substrates (C4 to C12), whereas it is only very slightly active in the presence of C16 substrate. These data emphasize the specificity of particular substrates of the mycobacterial FAS-II system.

4.1.4. MabA and ACP-Dependence?

The enzymatic complex containing hihA which we identified as the elongation system FAS-II, apart from its specificity for the C12-C18 substrates, has the property of being ACP-dependent. The ACP-dependence of the protein InhA is illustrated by its much more marked affinity for the substrates derived from ACP (the Km for octenoyl-ACP is 2 orders of magnitude smaller than that for the derivative of C8 CoA). Determining the preference of MabA for ACP derivatives requires the synthesis of these (non-commercial) derivatives and comparison of the kinetic constants with those- of the CoA derivatives. The KARs of plants are ACP-dependent, a property which was correlated to their belonging to a type II system. The numerous arguments in favour of MabA belonging to FAS-II strongly suggest the ACP-dependence of β-ketoacyl reductase.

Conclusion

After development of the overproduction of the protein MabA in Escherichia coli and purification, we carried out an enzymatic study of this protein. Thus, we showed that its catalytic activity corresponds to the NADPH-dependent reduction of β-ketoesters, which corresponds to one of the stages of the fatty acid elongation route. Determination of the activity of MabA in the presence of substrates with several chain lengths made it possible to show the preference of this enzyme for substrates of a size greater than or equal to 12 carbon atoms, in accordance with its potential involvement in a fatty acid elongation system. We therefore sought the protein MabA in the FAS-II enzymatic complex containing InhA, and studied the involvement of MabA in the elongation activity of this system.

5. Contribution of Molecular Modelling to the Study of the Protein MabA

Molecular modelling makes it possible to access a set of information concerning the structural characteristics of the protein, the architecture of the catalytic site, but also to assess the possibilities of interaction with ligands (substrates, inhibitors, affine molecules). The production of the three-dimensional model of the protein MabA is presented below.

5.1. Search for Proteins Having a High Sequence Similarity With MabA

A search for peptide sequences similar to that of MabA (M. tuberculosis) in data banks with Psi-blast software (Altschul et al., 1997) showed that β-ketoacyl-ACP reductases existed having a high level of identity with MabA (87%, 84%, 69%, respectively) in other mycobacterial species (avium, smegmatis, leprae). Proteins homologous to MabA, called FabG, are also present in other organisms, essentially bacteria (for example in Streptomyces ceolicolor, 57% identity) and plants. However, no β-ketoacyl-ACP reductase structure has ever been resolved. Producing a molecular model of MabA was therefore of interest in the study of FabG.

5.2. Production of the MabA Model

The structural modelling of MabA was carried out using the programme Modeller 4 (Sali & Blundell, 1993). The model is based on the structures of proteins crystallized in complex with NAD(P)(H) and having the highest level of identity and lowest probability score (E) with MabA. These “support” proteins, selected using Psi-blast software (Altschul et al., 1997) in the main protein structure data bank, the PDB (Protein Data Bank, (Berman et al., 2000)), are: PDB2HSD (34%/NAD); PDB1YBV (33%/NADPH); PDB2AE2 (29%/NADP); PDB1FMC (28%/NADH); PDB1CYD (28%/NADPH) and PDB1BDB (27%/NAD). These proteins, of very diverse origin, all catalyze either the reduction of a carbonyl (such as MabA), or the reverse reaction. The alignment of sequences used for the modelling was carried out by considering the well-preserved regions between MabA and the supports, on .the one hand, and between MabA and the other known β-ketoacyl-ACP reductases (FabG), on the other hand. In order to verify that the model is energetically stable, two programmes were used, TITO (Labesse & Momon, 1998) and Verify-3D (Luthy et al., 1992), which produced satisfactory scores.

The monomeric structure produced by the MabA model indicates that the protein belongs to the α/β structural superfamily, with six α helices and seven β strands. It should be noted that the β6-α6″ loop comprises two helices called α6 and α6′. MabA possesses a single domain, the topology of which is similar to Rossmann folding (β/α)₆ (Rossmann et al., 1974), typical of the dinucleotide-diphosphate-binding proteins (DDBP) (Persson et al., 1991). However, in contrast to the DDBP with two domains, there is no symmetry, since the helices of the C-terminal moiety (α4, α5) are longer than the secondary structures of the N-terminal part. These characteristics, as well as the presence of an additional strand (β7), are typical of the RED (Reductase/Epimerase/Dehydrogenase) proteins superfamily (Labesse et al., 1994). The presence of a single cysteine (C60), probably buried, in MabA excludes the possibility of formation of an intra- or inter-chain disulphide bond within the protein.

[symbol]

The bound NADPH cofactor is found in an extended conformation resting on the C-terminals ends of the β1-β5 strands which form a leaf. The β2 strand of the RED proteins which is involved in the binding of the ribose linked to the adenine of the cofactor, has, in the MabA sequence, the unit [* * * xxr] [symbol] , specific to NADP(H)-dependent enzymes (Labesse et al., 1994). This is in agreement with the enzymatic data showing the strict specificity of MabA for NADPH and indicates that the additional phosphate is probably important for the stabilization of the cofactor in satisfactory orientation for the catalysis. The positively charged residue R47, forming part of the unit [VAVTHR] of the strand β2, is probably involved in the interaction of the protein with the phosphate, by electrostatic bonds. [symbol]
*: hydrophobic residue, x: any amino acid. In capital and small letters the strictly preserved residues and those most frequently encountered, respectively.

As in the other RED proteins, the binding site of the substrate of MabA is probably delimited by the C-terminal ends of the strands β4, β5, β6, β7 and the helices α4, α5, α6 (α6, α6′, α6″) (FIG. 5.18; (Labesse et al., 1994)); the nicotinamide part of NADPH, involved in the ion exchanges, is oriented towards the bottom of the cavity. The residues of the active site which are very well preserved, and constitute in part the signature of RED proteins, are present in the catalytic site of MabA: the catalytic triad, S140,Y153, K157 and N112, T188.

5.3.Relation Between Structure and Function of MabA

According to the atomic coordinates of the MabA model, the single tryptophan (W145), situated at the level of the β5-α5 loop, is probably involved in the catalytic pocket. The latter appears very hydrophobic because of the involvement of the C-terminal arm (rich in hydrophobic residues) in the structure of this pocket on the one hand, and by the presence, in addition to W145, of residues such as I147 and F205, on the other hand. In the proteins FabG of other organisms and specific to short chain substrates, the latter two residues are replaced by more polar residues, Asn (for I147) and Thr, Gln or Asn (for F205). The specificity of MabA for long chain substrates is very probably linked with the hydrophobic character of the catalytic pocket which thus constitutes a favourable environment for receiving aliphatic long chains, a structure-function relation between the hydrophobicity and the size of the substrate-binding pocket and the affinity for long chain molecules has already been demonstrated for the protein InhA (Rozwarski et al., 1999), which also forms part of the REDs.

The superposition of the MabA model on the crystalline structure of InhA (ternary complex C16 InhA-NAD⁺-substrate, (Rozwarski et al., 1999); PDB1BVR) reveals that the substrate-binding pockets of the two proteins have similar sizes, in accordance with their affinity for substrates possessing similar chain lengths (C₁₂-C₂₄ for InhA, C₈-C₂₀ for MabA; [(Rozwarski et al., 1999); (Quémard et al., 1995)]. However, the binding pocket of the enoyl reductase InhA is still more hydrophobic than that of MabA, which could explain the slight shift in the specificity of substrates between InhA (maximum specific activity in the presence of C₁₆, (Quémard et al., 1995)) and MabA (maximum specific activity in the presence of C₁₂).

The alignment of MabA sequences with the support proteins and with all of the known proteins FabG indicates that the amino-terminal end is not preserved; this region “floats” to the outside of the protein and does not correspond to a defined secondary structure. This suggests that this domain of the protein can tolerate variations, and that it is not important for the function of the protein. Experimental data in agreement with this proposition are provided by study of the catalytic activity of H-MabA. The protein comprises an NH₂-terminal poly-histidine tag the presence of which does not seem to affect the catalytic activity.

5.4. Quaternary Structure of MabA

Due to their secondary structures and tertiary characteristics, all the RED proteins described are dimeric or tetrameric (dimer of dimers). The C-terminal region of MabA, corresponding to the α6-β7 loop and the β7 strand, has a very high similarity with the equivalent region of the known tetrameric REDs, in particular with that of PDB2HSD for which it was shown that this region was involved in the dimer-dimer interface of the heterotetramer (Persson et al., 1991). The second interface between two monomers in PDB2HSD involves the helices α4 and α5. The preservation in MabA of the C-terminal end and the presence of hydrophobic amino acids at the surface of the helices α4 and α5 suggest that MabA is tetrameric. These results are in agreement with the exclusion chromatography analysis, suggesting an equilibrium between the dimeric and tetrameric forms of MabA. Analysis of the monomer-monomer and dimer-dimer interfaces in a tetrameric model of MabA could make it possible to reinforce this conclusion.

5.5. Interaction With Antibiotics

It has been shown that the active form of the INH which inhibits the protein InhA would be an isonicotinoyl-NAD radical or anion (Rozwarski et al., 1998). These authors have suggested that the isonicotinoyl-NAD adduct is formed in the catalytic site of InhA, whilst Wilming and Johnsson have shown that its formation can occur in the absence of the enzyme (Wilming & Johnsson, 1999). Thus, doubt remains as to the exact effect of the INH on IhA in vivo. The superposition of the MabA model on the structure of the binary complex InhA-isonicotinoyl-NAD (PDB1ZID) shows that there is no incompatibility with the binding, in the active site of MabA, of molecules such as isoniazid or ethionamide. Similarly for the protein InhA, the isonicotinoyl-NADP adduct could a priori be fixed on MabA, once it is formed. However, in the case of the adduct being formed within the catalytic site, it cannot be foreseen whether the isoniazid would have an appropriate orientation and could interact with the cofactor NADPH. In all cases, an inhibition of the activity of MabA by INH must be verified biochemically, as the model does not allow a precise teaching on the topology of the lateral chains of the active site.

6. Inhibition of the Activity of MabA

We tested the effect of isoniazid on the β-ketoacyl reductase activity of MabA by adopting experimental conditions similar to those making it possible to observe an inhibition of the activity of InhA. The protein MabA (150 nM) is preincubated for 2 hours in the presence of 100 μM or 2 mM isoniazid, 100 μM NADPH and 1 μM MnCl₂. In the presence of 100 μM of INH, the activity of MabA, demonstrated in the presence of acetoacetyl-CoA, is inhibited by 44±3% compared with the control without INH and the addition of 2 mM of isoniazid, leads to an inhibition of 62±6%. The fact that total inhibition of the activity of MabA is not observed even in the presence of 2 mM isoniazid could be explained by a very slow oxidation of the isoniazid by the Mn³⁺ ions under the conditions used (in the absence of a catalyst such as KatG), and therefore a concentration in active form of the antibiotic which is not proportional to the starting concentration of isoniazid. This explanation assumes that MabA is inhibited by a mechanism similar to that described for InhA. It should be recalled that the inhibition mechanism of the protein InhA by isoniazid requires at a minimum the presence of the cofactor NADH, Mn²⁺ ions and molecular oxygen. The Mn²⁺ ions would be oxidized to Mn³⁺, which, in turn catalyze the oxidation of the isoniazid. Thus, we tested the effect of the absence of MnCl₂ or NADPH on the inhibition of MabA by INH. In the absence of MnCl₂ in the reaction, a non-significant reduction in the activity of the enzyme is observed, indicating that the Mn²⁺ ions are necessary in order to obtain an effect of the INH. Determination of the involvement of NADPH in the inhibition process of is more difficult to achieve, as preincubation of the protein MabA in the absence of this cofactor leads to a considerable reduction (−74%) in the activity after preincubation for 2 hours without antibiotics. It was therefore not possible to evaluate the involvement of NADPH in the inhibition by isoniazid.

Our results show that the activity of the protein MabA is inhibited by isoniazid, and suggest that the action mechanism of this antibiotic on MabA would cause the intervention of Mn²⁺ ions. Given the structure and function homology of the two proteins, it is probable that the inhibition mechanism is analogous to that of InhA, passing through the formation of an isonicotinoyl-NADP⁺ adduct.

In mycobacteria, a mutation or overexpression of the inhA gene leads to cross-resistance to the two antituberculous agents isoniazid and ethionamide (ETH). Ethionamide is probably also a prodrug since the inhibition of the protein InhA by this antibiotic in its native form has not been observed in vitro. However, the mode of activation of ETH is not known. We nevertheless tested the effect of ethionamide on the activity of InhA, under the experimental conditions of inhibition by INH, in the presence of MnCl₂ and NADH. In the presence of 100 μM of ETH, the activity of InhA is unchanged. The same result is obtained on MabA. It was not possible to test higher concentrations of antibiotics due to the strong absorbance that it has in the wavelength region used for the enzymatic tests. Nevertheless, the conditions for oxidation of INH in vitro adopted for the ethionamide test do not seem to be those required for the activation of ETH. The fact that the catalase-peroxidase KatG, which accelerates the oxidation of isoniazid is not the activator of ETH [(Johnsson et al., 1995); (Basso et al., 1996)] is in agreement with this conclusion. On the other hand, if oxidation of ETH is required for its action on its targets(s), the oxidation of a thioamide function proves more difficult than that of a hydrazide function (INH) and should require an oxidizing agent stronger than Mn³⁺ ions.

7. Conclusion and Discussion

Study of the three-dimensional structure of MabA by molecular modelling made it possible to show that the protein has a single domain, with a secondary structure of α/β type, and that it belongs to the RED structural superfamily (reductases/epimerases/dehydrogenases). The protein MabA possesses the specific unit of the proteins binding NADP(H) and a substrate-binding pocket the size and hydrophobicity of which promote the reception of long chain β-ketoesters. These structural data provided by the MabA model are in agreement with the biochemical results obtained previously. The MabA model indicates that the tryptophan (Trp) residue, situated at the level of the β5-α5 loop, would be involved in the substrate-binding pocket. Thanks to the uniqueness of this Trp residue, it was possible to carry out fluorescence spectroscopy experiments. They made it possible to validate the MabA model, confirming the involvement of Trp and at least one of the two Mets of the C-terminal end in the substrate-binding pocket on the one hand, and the specificity of MabA for long chain substrates on the other hand. The superposition of the MabA model with the crystalline structure of InhA (in complex with NAD⁺ and the C16 substrate, (Rozwarski et al., 1999) reveals that the two proteins have substrate binding sites of equivalent size and more hydrophobic than their homologues of other organisms, involved in a synthesis de novo. This confirms the hypothesis of co-involvement of MabA and InhA in the same fatty acids elongation complex.

The MabA model suggests that the protein in solution is tetrameric, which is in agreement with the result of the gel filtration experiments, having suggested that there was, under the experimental conditions tested, a dimer-tetramer equilibrium of MabA. However, the combination of MabA with InhA, each in the tetrameric form in the FAS-II complex, is incompatible with the estimated size of the system. Thus, as InhA and MabA have similar topologies, it could be postulated that these two proteins form a heterotetramer within the FAS-II complex. In order to test this hypothesis, the molecular modelling of an MabA-InhA heterotetramer complex, using tetrameric RED proteins as supports, can be carried out. In order to confirm the possibility that InhA and MabA can be combined in complex, chemical bridging between the two proteins can be attempted in the presence of their respective cofactors.

Knowledge of the three-dimensional organization given by the model suggests a possible interaction between MabA and isoniazid. We were able to show, by enzymatic studies, that the activity of MabA was effectively inhibited in vitro by this antibiotic and that the inhibition mechanism of MabA is probably comparable with that described for the protein InhA.

II) Material and Methods

M.1. Overexpression of the mabA Gene in E. Coli

M.1.1. Construction of the pET-15b::mabA Expression Plasmid

The mabA gene of M. tuberculosis was cloned between the NdeI and Xho sites of the pET-15b plasmid, downstream of a sequence coding for 6 histidines.

M. 1.2. Transformation of BL21(λDE3) E. coli by pET-15b::mabA Plasmid

After preparation of competent bacteria of E. coli BL21(λDE3) (Sambrook et al., 1989), an aliquot (100 μl) is incubated in the presence of the pET-15b::mabA plasmid (39 ng) for 30 minutes in ice. The transformation is carried out by thermal shock (90 seconds at −42° C., then 2 minutes in ice). LB medium is then added and the suspension is incubated for 45 minutes at 37° C. under stirring (250 rpm) before being plated on LB-agar dishes containing 50 μg/ml of carbenicillin. Incubation at 37° C. for approximately 18 hours makes it possible to obtain medium to large-sized colonies.

M.1.3. Induction of the Expression of the Target Gene

Four medium-sized colonies are used for seeding four cultures of 50 ml in LB medium+carbenicillin. The turbidity of the medium is measured by spectrophotometry at 600 nm hourly until the optical density reaches 0.8 (middle of the exponential growth phase), i.e. after incubation for approximately 4 hours. The expression of the mabA gene is then induced with 0.8 mM of IPTG for 2 hours at 37° C., then verified by SDS-PAGE. An aliquot of non-induced culture is preserved and will serve as negative control of the induction.

M.1.4. Verification of the Overexpression

Once the expression of mabA is induced, an aliquot of 100 μl of culture is analyzed in order to check the expression of the gene. After centrifugation (5 minutes at 12000 g), the bacterial pellet is taken up in charge buffer (Laemnmli, 1970) in order to be applied to 12% polyacrylamide gel under denaturing conditions.

M.1.5. Small-Scale Protein MabA Solubility Test

The bacteria (10 ml) are collected by centrifugation for 5 minutes at 3000 g, at 4° C. The pellet is resuspended in potassium phosphate buffer (100 mM, pH 7.2) in 1/20 of the initial volume of the culture. The suspension is sonicated using a microprobe (Vibracell, Bioblock), using four pulses of 10 seconds interspersed with recovery times of 40 seconds (duty cycle: 60%, microtip limit: 5). The total extract obtained is centrifuged for 5 minutes at 12000 g, at 4° C. The presence of the protein MabA in the fractions corresponding to the total (soluble) supernatant and (insoluble) pellet is analyzed by SDS-PAGE (12% polyacrylamide).

M.2. Purification of MabA

All the stages are carried out at a low temperature (0-4° C.), in order to reduce the action of the proteases.

M.2.1. Preparation of the Bacterial Lysate

All of the cultures (4×50 ml) are collected by centrifugation (15 minutes at 16000 g, at 4° C.) then washed (50 mM potassium phosphate buffer, pH 7.8). The pellet obtained (0.9 g/200 ml of culture) is taken up in 9 ml of lysis buffer (50 mM potassium phosphate buffer, pH 7.8 containing 500 mM of NaCl and 5 mM of imidazole). Before freezing the suspension at −70° C. (overnight), a mixture of protease inhibitors (0.113 mg/ml, see below) and lysozyme (0.5 mg/ml) are added to it. The suspension is thawed, under gentle stirring, at ambient temperature, then treated with DNaseI (5 μg/ml) and RNaseA (10 μg/ml) in the presence of 10 mM MgCl₂ for 15 minutes at 4° C., under gentle stirring. The whole bacteria and the debris are eliminated by centrifugation (15 minutes at 3000 g, at 4° C.). A last ultracentrifugation at 44000 g, 45 minutes at 4° C. , makes it possible to eliminate any insoluble material. 10% (v/v) of glycerol is added to the supernatant (clarified lysate) before being loaded on the column.

Mixture of Protease Inhibitors:

| | |
|---|---|
| leupeptin (chymotrypsin inhibitor): | 0.0023 g/l |
| soybean (reversible trypsin inhibitor): | 0.02 g/l |
| TLCK (irreversible trypsin inhibitor): | 0.0518 g/l |
| Aprotinin | 0.0016 g/l |
| Pepstatin (pepsin-like inhibitor) | 0.0011 g/l |
| PMSF (irreversible chymotrypsin inhibitor) | 0.0362 g/l |

Note:
In these experiments, the EDTA (metal-dependent protease inhibitor) is omitted from the mixture of protease inhibitors, because of its ability to chelate nickel ions during purification on an Ni-NTA column.

M.2.2. Purification of H-MabA in a Minicolumn

In an empty minicolumn (total volume 7.5 ml in polypropylene, Polylabo), 500 μl of Ni-NTA agarose resin (QIAGEN) (i.e. 1 ml of 50% suspension) are washed with 4 times 25 ml of lysis buffer*. Four ml of clarified bacterial lysate (approximately 15 mg of total protein) are incubated with the Ni-NTA-resin under gentle stirring, for 1 hour at 4° C. The material not bound to the resin is recovered by decantation, then by “washings” with 32 CV (column volumes) of lysis buffer. The remainder of the contaminant proteins is eluted with 8 CV of buffer with 50 mM imidazole. The protein MabA is eluted by 8 CV of buffer with 175 mM imidazole. The resin is then cleaned with 10 CV of buffer with 250 mM imidazole and recovered directly in pure glycerol in order to have 50% (v/v) of final glycerol. These precautions are necessary in order to avoid the precipitation of MabA at the column outlet.

Note: all the buffers used here contain 50 mM of potassium phosphate pH 7.8 and 500 mM of NaCl.

lysis buffer: 50 mM potassium phosphate buffer, pH 7.8 containing 500 mM NaCl and 5 mM of imidazole.

M.2.3. Dialysis of the Solution of Purified H-MabA

The solution of protein MabA after purification, recovered in 50% (v/v) glycerol, is dialyzed twice for 1 hour against 40 volumes of 50 mM potassium phosphate buffer pH 7.2 containing 50% (v/v) of glycerol, at 4° C., in a dialysis tube (cutoff threshold 8-10 kDa, Spectra/Por, Spectrum) previously boiled in a solution of 1 mM EDTA in order to eliminate traces of heavy metals, then rinsed with osmosis-purified water. The dialysate is then aliquoted and stored at −20° C.

M.2.4. Determination of the Quantity of Purified Protein by U.V. Spectroscopy

We estimated the concentration of the solutions of protein purified according to the Beer-Lambert law (OD=ε1C*) with its theoretical molar extinction coefficient and its absorbance at 280 nm.

ε: molar extinction coefficient, 1: length of the optical path and C: molar concentration.

Theoretical Determination of the Molar Extinction Coefficient (Deduced From the Protein Sequence)

The molar extinction coefficients (MEC) of the proteins are calculated according to Gill and Von Hippel (Gill & von Hippel, 1989) (in the presence of 6M guanidine chloride at pH 6.5). The relation is then the following:

MEC=(a * ETyr)+(b * ETrp)+(c * ECys), where a, b and c are respectively the number of residues, and Eaa their molar extinction coefficients. At 280 nm, they are respectively equal to: ETyr=1280 ETrp=5690 ECys=120

The MEC (ε) of MabA at 280nm is 9530 M⁻¹cm⁻¹ and does not take account of the single Cys residue.

M3. Study of the Properties of MabA

M.3.1. Determination of the Mass of H-MabA by Electrospray Ionization/Mass Spectrometry (ESI/MS)

A pellet of 2 mg of purified protein H-MabA, precipitated in buffer without glycerol, is washed 5 times with water (centrifugation for 5 minutes at 12000 g). 200 μl of acetonitrile/water mixture (50/50)+0.1% (v/v) TFA are added, then the whole mixture is vortexed and centrifuged for 2 minutes at 12000 g. An equal volume of a methanol/water mixture (50/50)+0.5% (v/v) acetic acid is added to an aliquot of the supernatant, the mixture is vortexed then kept for 2 hours at 4° C. before being centrifuged for 2 minutes at 12000 g. 60 μl of the supernatant are introduced into the source of the spectrometer via a syringe pump (HARVARD), at a flow rate of 5 μl/min, in order to be analyzed by electrospray ionization/mass spectrometry (ESI/MS) on a Finnigan MAT device (TSQ 700). The parameters of the ESI source correspond to a 5 kV power supply, a temperature of the intermediate capillary of 250° C. and 40 psi for the nitrogen (nebulization gas).

M3.2. Determination of Native Size by Gel Filtration

FPLC experiments were carried out with the BioCAD SPRINT system (PerSeptive Biosystems, Cambridge, Mass.). A Sephacryl S-100 HR column (HiPrep™ 16/60 Sephacryl High Resolution, Pharmacia) was equilibrated with 1 CV of 50 mM potassium phosphate buffer, pH 6.8 containing 100 mM NaCl. Five standard proteins of known molecular masses, diluted in this same buffer, were applied to the column (0.5 to 1 mg of each protein): alcohol dehydrogenase (150 kDa), bovine serum albumin (BSA, 67 kDa), ovalbumin (43 kDa), carbonic anhydrase (29 kDa) and RibonucleaseA (RNaseA, 13.7 kDa). Two successive elutions were carried out with a different combination of 3 standard proteins in order to obtain a better resolution of the peaks, and the profiles at 280 nm were superimposed. The calibration curve was obtained by plotting the elution volume of each standard protein as a function of the logarithm of the molecular mass. A solution of H-MabA at 1.1 mg/ml (0.66 mg of loaded protein) is applied to the column and eluted sunder the same conditions as the standard proteins. The molecular mass of H-MabA is estimated with-reference to the calibration curve.

M.4. Enzymatic Study of MabA

M.4.1. Calibration of the Solutions of Reagents

Determination of the kinetic parameters for the different substrate requires enzymatic test conditions which can be reproduced from one manipulation to another. The concentrations of the solutions of β-ketoester substrate of CoA and cofactor (NADPH) are therefore determined before use. The reagent to be calibrated (for example β-ketoester of CoA) is added at a concentration considerably lower than that of the second substrate (NADPH). The reaction is triggered with a sufficient enzyme concentration in order to obtain the rapid use of the substrate in limiting concentration. The difference of OD₃₄₀ observed makes it possible to deduce the real concentration of this β-ketoester substrate of CoA in the reaction.

M.4.1.2. Description of the Enzymatic Test

The catalytic activity of purified MabA was demonstrated by spectrophotometry in the presence of acetoacetyl-CoA and NADPH.

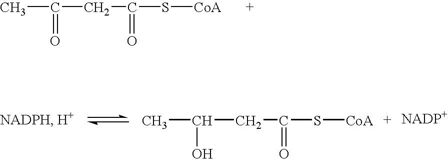

The kinetics of the β-ketoacyl reduction reaction are monitored by measuring the absorbance at 340 nm over time, which decreases with the oxidation of the NADPH. The enzymatic reaction is carried out in a fmal volume of 1 ml (in a quartz cuvette, optical path 1 cm). The spectrophotometer (UVIKON 923, Bio-Tek Kontron Instruments) is connected to a thermostatically-controlled bath making it possible to regulate the temperature of the cuvette at 25° C. A base line is carried out in the absence of enzyme. The reaction mixture comprises 80 mM of sodium phosphate buffer, and variable concentrations of NADPH and β-ketoacyl-CoA. The reaction is triggered by the addition of the enzyme (36 nM to 144 nM). The measurements are carried out over 3 to 5 minutes.

The Kₘ for the NADPH was determined at concentrations of coenzyme varying from 5 to 200 μM and at a fixed concentration (460 μM) of acetoacetyl-CoA. The Kₘs for the β-ketoacyl-CoA were determined at a fixed concentration, 100 μM, of NADPH. A concentration above 100 μM led to too much noise at the level of the measurements. It was verified, moreover, that this concentration was saturating.

The Kₘ and Vₘₐₓ for the β-ketoacyl-CoAs were measured at the following concentrations: for the acetoacetyl-CoA (C₄), 100-8570 μM; for the β-ketooctanoyl-CoA (C₈), 4-160 μM; for the β-ketododecanoyl-CoA (C₁₂), 2-32 μM. For the β-ketohexadecanoyl-CoA and the ⊕-ketoeicosanoyl-CoA (C₂₀), problems of inhibition by the substrate made it possible to determine the kinetic parameters and the reaction velocity was compared at a fixed concentration of 2 μM.

At least two series of experimental points were produced for each kinetic parameter. The accuracy of these points was verified graphically by “double inverse” representation, 1/v=f(1/[S]) (equation (1)). The kinetic parameters were then determined graphically according to the Hanes representation [S]/v=f([S]) (equation (2)) or by calculation according to the least error squares method, with GraphPad Prism software Version 2.01.

$$\begin{matrix}\frac{1}{V} = \frac{{K}_{m}}{{V}_{max}} \cdot \frac{1}{S} + \frac{1}{{V}_{max}} ( Lineweaver \text{-} Burk ) & Equation ( 1 ) \\ \frac{S}{ν} = \frac{S}{V max} + \frac{Km}{V max} ( Hanes ) & Equation ( 2 )\end{matrix}$$

III) Mutagenesis of the Protein MabA and Optimum Purification Methods for the Protein MabA, and the Proteins MabA C(60)V, MabA S(144)L and MabA C(60)V/S(144)L

1) Mutagenesis of the Protein MabA

The mutant MabA C(60)V was obtained by site-specific mutagenesis after carrying out an inverse PCR. The nucleotide primers were chosen so as to modify codon 60 of the mabA gene, namely replacement of TGT (cysteine) by GTT (valine). The pET15b::mabA plasmid was used as support for the PCR amplification by DNA polymerase PfuTurbo (Stratagene, USA).

The PCR products were digested with the endonuclease Dpn1 in order to select the plasmids comprising the mutated gene. The mutated gene was entirely sequenced in order to verify the absence of secondary mutation. The plasmid carrying the mabA C(60)V gene (pET15b::mabA C(60)V) was then used to transform the superproducing strain BL21(DE3).

The mutants MabA C(60)V/S(144)L and MabA S(144)L were obtained according to the same method as previously.

2) Purification of the Proteins MabA, MabA C(60)V, and MabA C(60)V/S(144)L

Four cultures of 50 ml in LB medium+carbenicillin are carried out. The turbidity of the medium is measured by spectrophotometry at 600 nm until the optical density reaches 0.8 (middle of the exponential growth phase), i.e. after incubation for approximately 4 hours. The expression of the mabA gene is then induced with 0.8 mM of IPTG for 2 hours at 37° C., then verified by SDS-PAGE.

All of the cultures (4×50 MnCl) are collected by centrifugation (15 minutes at 16000 g, at 4° C.) then washed. The pellet obtained is taken up in 4 ml of lysis buffer (see below). Before freezing the suspension at −80° C. (overnight), a mixture of protease inhibitors (0.113 mg/ml) and lysozyme (0.5 mg/ml) are added to it. The suspension is thawed under gentle stirring at ambient temperature, then treated with DNaseI (5 μg/ml) and RNaseA (10 μg/ml) in the presence of 10 mM MgCl₂ for 15 minutes at 4° C., under gentle stirring. The whole bacteria and the debris are eliminated by centrifugation (15 minutes at 3000 g, at 4° C.). A last ultracentrifugation at 44000 g, 15 minutes at −4° C., makes it possible to eliminate any insoluble material. According to the case, before being loaded onto the column, the supernatant (clarified lysate) can be complemented with either 10% (v/v) of glycerol (protein for kinetic studies, or for crystallography of the least stable proteins), or 400 μM NADP⁺ (crystallographic study of the MabA-NADP complex).

Four ml of clarified bacterial lysate (approximately 30 mg of total proteins) are added to an Ni-NTA agarose column (500 μl, QIAGEN). The material not bound to the resin is recovered by “washings” with buffer with 5 mM then 50 mM imidazole. The protein MabA is eluted with the elution buffer. When the phosphate buffer is used, the protein is recovered directly in 50% (v, v) final glycerol, in order to avoid precipitation. For the crystallography, the protein is concentrated to 10-15 mg/ml by ultrafiltration.

Buffers Used:

Proteins for Kinetic Studies:

Lysis buffer: 50 mM potassium phosphate, pH 7.8 containing 500 mM of NaCl, 5 mM of imidazole

Washing buffers: 50 mM potassium phosphate, pH 7.8 containing 500 mM of NaCl, 5 and 50 mM of imidazole

Elution buffer: 50 mM potassium phosphate, pH 7.8 containing 500 mM of NaCl, and 175 mM of imidazole.

Proteins for Crystallography Studies:

Lysis buffer: 50 mM Tris buffer, pH 8.0, supplemented with 300 mM LiSO₄ and 5 mM imidazole;

or: 50 mM Tris buffer, pH 8.0, supplemented with 300 mM KCl and 5 mM imidazole.

Washing buffers: 50 mM Tris buffer, pH 8.0, supplemented with 300 mM LiSO₄ and 5 or 50 mM imidazole;

or: 50 mM Tris buffer, pH 8.0, supplemented with 300 mM KCl and 5 or 50 mM imidazole.

Elution buffer: 20 mM MES buffer, pH 6.4, 300 mM LiSO₄ and 175-750 mM imidazole;

or: 20 mM PIPES buffer, pH 8.0, supplemented with 300 mM KCl and 175-750 mM imidazole.

Note: 1 mM DTT is added to these buffers in the case of the wild-type protein.

(MES=2-[N-morpholino]ethane sulphonic acid; PIPES=piperazine-N,N′-bis[2-ethane sulphonic acid])

3) Peptide Sequences of the Proteins Obtained and Nucleotide Sequences Coding for These Proteins

Peptide sequence of the wild-type protein MabA (FabG1) of M. tuberculosis H37Rv in fusion with a poly-His tag (in bold):

MGSSHRHHHH SSQLVPRGSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMTFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

Peptide sequence of the protein MabA C60V (mutation in bold) in fusion with a poly-His tag (in bold):

MQSSHHHHHH SSGLVPRGSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSTARE LSKANVTANV
VAPGYIDTDM TRALDERIQQ GALQFIPAKR VGTPAEVAGV VSFLASEDAS
YISGAVIPVD GGMGMGH

Peptide sequence of the protein MabA C60V/S144L (mutations in bold) in fusion with a poly-His tag (in bold):

MGSSHHHHHH SSGLVPRGSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH

Nucleotide sequence of the wild-type mabA (fabG1) gene of M. tuberculosis strain H37Rv, in fusion with a sequence coding for a poly-Histidine tag (in capital letters):

ATGGGCAGCAGCCATCATCATCATCATCACAGCAGCGGCCTGGTGCCGCG
CGGCAGCCATatgactgccacagccactgaaggggccaaacccccattcg
tatcccgttcagtcctggttaccggaggaaaccgggggatcgggctggcg
atcgcacagcggctggctgccgacggccacaaggtggccgtcacccaccg
tggatccggagcgccaaaggggctgtttggcgtcgaatgtgacgtcaccg
acagcgacgccgtcgatcgcgccttcacggcggtagaagagcaccagggt
ccggtcgaggtgctggtgtccaacgccggcctatccgcggacgcattcct
catgcggatgaccgaggaaaagttcgagaaggtcatcaacgccaacctca
ccggggcgttccgggtggctcaacgggcatcgcgcagcatgcagcgcaac
aaattcggtcgaatgatattcataggttcggtctccggcagctggggcat
cggcaaccaggccaactacgcagcctccaaggccggagtgattggcatgg
cccgctcgatcgcccgcgagctgtcgaaggcaaacgtgaccgcgaatgtg
gtggccccgggctacatcgacaccgatatgacccgcgcgctggatgagcg
gattcagcagggggcgctgcaatttatcccagcgaagcgggtcggcaccc
ccgccgaggtcgccggggtggtcagcttcctggcttccgaggatgcgagc
tatatctccggtgcggtcatcccggtcgacggcggcatgggtatgggcca
c

Nucleotide sequence of the mabA (fabG1) C60V gene (mutated codon in bold) of M. tuberculosis strain H37Rv, in fusion with a sequence coding for a poly-Histidine tag (in capital letters):

ATGGGCAGCAGCCATCATCATCATCATCACAGCAGCGGCCTGGTGCCGCG
CGGCAGCCATatgactgccacagccactgaaggggccaaacccccattcg
tatcccgttcagtcctggttaccggaggaaaccgggggatcgggctggcg
atcgcacagcggctggctgccgacggccacaaggtggccgtcacccaccg
tggatccggagcgccaaaggggctgtttggcgtcgaaGTTgacgtcaccg
acagcgacgccgtcgatcgcgccttcacggcggtagaagagcaccagggt
ccggtcgaggtgctggtgtccaacgccggcctatccgcggacgcattcct
catgcggatgaccgaggaaaagttcgagaaggtcatcaacgccaacctca
ccggggcgttccgggtggctcaacgggcatcgcgcagcatgcagcgcaac
aaattcggtcgaatgatattcataggttcggtctccggcagctggggcat
cggcaaccaggccaactacgcagcctccaaggccggagtgattggcatgg
cccgctcgatcgcccgcgagctgtcgaaggcaaacgtgaccgcgaatgtg
gtggccccgggctacatcgacaccgatatgacccgcgcgctggatgagcg
gattcagcagggggcgctgcaatttatcccagcgaagcgggtcggcaccc
ccgccgaggtcgccggggtggtcagcttcctggcttccgaggatgcgagc
tatatctccggtgcggtcatcccggtcgacggcggcatgggtatgggcca
c

Nucleotide sequence of the mabA (fabG1) C60V/S144L gene (mutated codons in bold) of M. tuberculosis strain H37Rv, in fusion with a sequence coding for a poly-Histidine tag (in capital letters):

ATGGGCAGCAGCCATCATCATCATCATCACAGCAGCGGCCTGGTGCCGCG
CGGCAGCCATatgactgccacagccactgaaggggccaaacccccattcg
tatcccgttcagtcctggttaccggaggaaaccgggggatcgggctggcg
atcgcacagcggctggctgccgacggccacaaggtggccgtcacccaccg
tggatccggagcgccaaaggggctgtttggcgtcgaaGTTgacgtcaccg
acagcgacgccgtcgatcgcgccttcacggcggtagaagagcaccagggt
ccggtcgaggtgctggtgtccaacgccggcctatccgcggacgcattcct
catgcggatgaccgaggaaaagttcgagaaggtcatcaacgccaacctca
ccggggcgttccgggtggctcaacgggcatcgcgcagcatgcagcgcaac
aaattcggtcgaatgatattcataggttcggtctccggcCTCtggggcat
cggcaaccaggccaactacgcagcctccaaggccggagtgattggcatgg
cccgctcgatcgcccgcgagctgtcgaaggcaaacgtgaccgcgaatgtg
gtggccccgggctacatcgacaccgatatgacccgcgcgctggatgagcg
gattcagcagggggcgctgcaatttatcccagcgaagcgggtcggcaccc
ccgccgaggtcgccggggtggtcagcttcctggcttccgaggatgcgagc
tatatctccggtgcggtcatcccggtcgacggcggcatgggtatgggcca
c

4) Enzymatic Properties

Measurements of the enzymatic kinetics carried out with MabA are the following: acetoacetyl-CoA (C₄:Kₘ=1530±81 μM, k_{cat}=1.9±0.0 s⁻¹), β-ketooctanoyl-CoA (C₈: Kₘ=70±8 μM, k_{cat}=3.5±0.0 s⁻¹), β-ketododecanoyl-CoA (C₁₂: Kₘ=8.3±0.8 μM, k_{cat}=4.3±0.2 s⁻¹).

5) Crystallographical Study

The atomic coordinates of the three-dimensional structure of the crystals of the protein MabA are represented in FIG. 1, said crystals moreover having the following characteristics:

cell parameters:
a=81.403 angströms, b=116.801 angströms, c=52.324 angströms,

α=β=90.00°, γ=122.30°,

space group: C2,

maximum diffraction=2.05 angströms.

The atomic coordinates of the three-dimensional structure of the crystals of the protein C(60)V are represented in FIG. 2, said crystals moreover having the following characteristics:

cell parameters:
a=82.230 angströms, b=118.610 angströms, c=53.170 angströms,
α=β=90.00°, γ=122.74°,

space group: C2,

maximum diffraction=2.6 angströms.

The atomic coordinates of the three-dimensional structure of the crystals of the protein C(60)V/S(144)L are represented in FIG. 3, said crystals moreover having the following characteristics:

cell parameters:
a=81.072 angströms, b=117.022 angströms, c=53.170 angströms,
α=β=90.00°, γ=122.42°,

space group: C2,

maximum diffraction=1.75 angströms.

### BIBLIOGRAPHIC REFERENCES

Altschul, S. F., Madden, T. L., Schaffer, A. A., Zhang, J., Zhang, Z., Miller, W. & Lipman, D. J. (1997). Gapped BLAST and PSI-BLAST: a new generation of protein database search programs. Nucleic Acids Res. 25, 3389-3402.
Baneijee, A., Dubnau, E., Quemard, A., Balasubramanian, V., Um, K. S., Wilson, T., Collins, D., de Lisle, G. & Jacobs, W. R. J. (1994). inhA, a gene encoding a target for isoniazid and ethionamide in Mycobacteriurh tuberculosis. Science.263, 227-30.
Barnes, P. F., Bloch, A. B., Davidson, P. T. & Snider, D. E. (1991). Tuberculosis in patients with immunodeficiency virus infection. N. Engl. J. Med. 234, 1644-50.
Basso, L. A., Zheng, R. & Blanchard, J. S. (1996). Kinetics of inactivation of WT and C243S mutant of Mycobacterium tuberculosis enoyl reductase by activated isoniazid. J. Am. Chem. Soc. 118, 113014-11302.
Berman, H. M., Westbrook, J., Feng, Z., Gilliland, G., Bhat, T. N., Weissig, H., Shindyalov, I. N. & Boume, P. E. (2000). The protein data bank. Nucleic Acids Res. 28, 235-242.
Birge, C. H. & Vagelos, P. R. (1972). Acyl carrier protein. Purification and properties of β-hydroxyacyl acyl carrier protein dehydrase. J. Biol. chem. 247, 4930-4938.
Caughey, I. & Kekwick, R. O. (1982). The Characteristics of Some Components of the Fatty Acid Synthetase System in the Plastids from the Mesocarp of Avocado (Persea americana) Fruit. Eur. J. Biochem. 123, 553-561.
Coakley, W. T., Brown, R. C. & James, C. J. (1973). The Inactivation of Enzymes by Ultrasonic Cavitation at 20 kHz. Arch. Biochem. Biophys. 159, 722-729.
Cohn, D. L., Bustreo, F. & Raviglione, M. C. (1997). Drug-resistant tuberculosis: review of the worldwide situation and the WHO/IUATLD global surveillance project. Clinical Infectious Diseases. Clin Infect Dis 24 (Suppl 1), S121-S130.
Cole, S. T., Brosch, R., Parkhill, J., Garnier, T., Churcher, C., Harris, D., Gordon, S. V., Eiglmeier, K., Gas, S., Barry, C. E., Tekaia, F., Badcock, K., Basham, D., Brown, D., Chillingworth, T., Connor, R., Davies, R., Devlin, K., Feltwell, T., Gentles, S., Hamlin, N., Holroyd, S., Homsby, T., Jagels, K. & Barrell, B. G. (1998). Deciphering the biology of Mycobacterium tuberculosis from the complete genome sequence. Nature 393, 537-44.
Constantinides, P. P. & Steim, J. M. (1985). Physical properties of fatty acyl-CoA. Critical micelle concentrations and micellar size and shape. J. Biol Chem 260, 7573-7580.
Dolin, P. J., Raviglione, M. C. & Kochi, A. (1994). Global tuberculosis incidence and mortality during 1990-2000. Bull World Health Organ 72, 213-220.
Gill, S. C. & von Hippel, P. H. (1989). Calculation of protein extinction coefficients from amino acid sequence data [published erratum appears in Anal Biochem 1990 September; 189(2):283]. Anal Biochem 182, 319-26.
Johnsson, K., King, D. S. & Shultz, P. G. (1995). Studies on the mechanism of action of isoniazid and ethionarnide in the chemotherapy of tuberculosis. J. Am. Chem. Soc. 117, 5009-10.
Kikuchi, S., Takeuchi, T., Yasui, M., Kusaka, T. & Kolattukudi, P. E. (1989). A very long-chain fatty acid elongation system in Mycobacterium avium and a possible mode of action of isoniazid on the system. Agric. Biol. Chem. 53, 1689-98.
Labesse, G. & Momon, J. (1998). Incremental threading optimization (TITO) to help alignment and modelling of remote homologues. Bioinformatics 14, 206-211.
Labesse, G., Vidal-Cros, A., Chomilier, J., Gaudry, M. & Momon, J.-P. (1994). Structural comparisons lead to the definition of a new superfamily of NAD(P)(H)-accepting oxidoreductases: the single-domain reductases/epimerases/dehydrogenases (the RED family). Biochem. J. 304, 95-99.
Laenmili, U. K. (1970). Cleavage of structural proteins during the assembly of the head of bacteriophage T4. Nature 227, 680-685.
Luthy, R., Bowie, J. U. & Eisenberg, D. (1992). Assessment of protein models with three-dimensional profiles. Nature 356, 83-85.
Persson, B., Kpook, M. & J\hat{0}rnvall, H. (1991). Characteristics of short-chain alcohol dehydrogenases and related enzymes. Eur. J. Biochem. 200, 537-543.
Phillips, T. A., Van Bogelen, R. A. & Neidhardt, F. C. (1984). Ion gene product of Escherichia coli is a heat-shock protein. J. Bacteriol. 159, 283-287.
Quémard, A., Lacave, C. & Laneelle, G. (1991). Isoniazid inhibition of mycolic acid synthesis by cell extracts of sensitive and resistant strains of Mycobacterium aurum. Antimicrob. Agents Chemother. 35, 1035-9.
Quemard, A., Mazeres, S., Sut, A., Laneelle, G. & Lacave, C. (1995). Certain properties of isoniazid inhibition of mycolic acid synthesis in cell-free systems of M. aurum and M. avium. Biochim Biophys Acta 1254, 98-104.
Quemard, A., Sacchettini, J. C., Dessen, A., Vilcheze, C., Bittman, R., Jacobs, W. R. J. & Blanchard, J. S. (1995). Enzymatic characterization of the target for isoniazid in Mycobacterium tuberculosis. Biochemistry 34, 8235-41.
Rawlings, M. & Cronan, J. J. E. (1992). The gene encoding Escherichia coli acyl carrier protein lies within a cluster of fatty acid biosynthetic genes. J. Biol. Chem. 267, 5751-5754.
Rossmann, M. G., Moras, D. & Olsen, K. W. (1974). Chemical and biological evolution of nucleotide-binding protein. Nature 250, 194-199.
Rozwarski, D. A., Vilcheze, C., Sugantino, M., Bittman, R. & Sacchettini, J. C. (1999). Crystal structure of the Mycobacterium tuberculosis enoyl-ACP reductase, InhA, in complex with NAD+ and a C16 fatty acyl substrate. J. Biol. Chem. 274, 15582-9.
Sali, A. & Blundell, T. L. (1993). Comparative protein modelling by satisfaction of spatial restraints. J. Mol. Biol. 234, 779-815.
Sambrook, J., Fritsch, E. F. & Maniatis, T. (1989). Molecular cloning: a laboratory manual. Cold Spring Harbor, N.Y.: Cold Spring Harbor laboratory Press.
Schulz, H. & Wakil, S. J. (1971). Studies on the mechanism of fatty acid synthesis. XXV. On the mechanism of beta-ketoacylacyl carrier protein reductase from Escherichia coli. J. Biol. Chem. 246, 1895-1901.
Sheldon, P. S., Kekwick, R. G. O., Sidebottom, C., Smith, C. G. & Slabas, A. R. (1990). 3-Oxoacyl-(acyl-carrier protein) reductase from avocado (Persea americana) fruit mesocarp. Biochem. J. 271, 713-720.
Sheldon, P. S., Kekwick, R. G. O., Smith, C. G., Sidebottom, C. & Slabas, A. R. (1992). 3-Oxoacyl-[ACP] reductase from oilseed rape (Brassica napus). Biochim. Biophys. Acta. 1120, 151-159.
Shen, Z. & Byers, D. M. (1996). Isolation of Vibrio harveyi acyl carrier protein and the fabG, acpP, and fabF genes involved in fatty acid biosynthesis. J. Bacteriol. 178, 571-573.
Shimakata, T. & Stumpf, P. K. (1982). Purification and Characterizations of β-Ketoacyl-[Acyl-Carrier-Protein] Reductase, β-Hydroxyacyl-[Acyl-Carrier-Protein] Dehydrase, and Enoyl-[Acyl-Carrier-Protein] Reductase from Spinacea oleracea Leaves. Arch. Biochem. Biophys. 218, 77-91.
Studier, F. W. & Moffatt, B. A. (1986). Use of bacteriophage T7 RNA polymerase to direct selective high-level cloned genes. J. Mol. Biol. 189, 113-130.
Studier, F. W., Rosenberg, A. H., Dunn, J. J. & Dubendorff, J. W. (1990). Use of T7 RNA polymerase to direct expression of cloned genes. Meth. Enzymol. 185, 60-89.
Takayama, K., Wang, L. & David, H. L. (1972). Effect of isoniazid on the in vivo mycolic acid synthesis, cell growth, and viability of Mycobacterium tuberculosis. Antimicrob Agents Chemother 2, 29-35.
Weeks, G. & Wakil, S. J. (1968). Studies on the mechanism of fatty acid synthesis 18. Preparation and general properties of the enoyl acyl carrier protein reductases from Escherichia coli. J. Biol. Chem. 243, 1180-1189.
Winder, F. G. & Collins, P. B. (1970). Inhibition by isoniazid of synthesis of mycolic acids in Mycobacterium tuberculosis. J. Gen. Microbiol. 63, 41-48.

1-40. (canceled)

41. Protein MabA, alsocalled protein FabG1, recombinant in purified form, or recombinant proteins derived from the protein: MabA by mutation of one or more amino acids, said derived proteins being in purified form, and having a NADPH-dependent β-ketoacyl reductase activity.

42. Purified recombinant protein MabA according to claim 41, said protein being a protein of mycobacteria.

43. A method for producing the recombinant protein MabA or derived recombinant proteins in purified form according to claim 42, as by transforming strains of E. coli with a plasmid containing a sequence comprising the gene coding for the protein MabA, or comprising a sequence coding for a protein derived from MabA, followed by a purification stage during which:
the abovementioned recombinant E. coli bacteria are washed in a washing buffer, then taken up in a lysis buffer, and lysed by a freeze/thaw cycle in the presence of protease inhibitors and lysozyme,
after treatment by DNAse I and RNAse A, in the presence of MgCl₂, and centrifugation, the lysis supernatant of the bacteria obtained in the preceding stage, to which 10% (v/v) of glycerol, or 400 μM of NADP⁺ is added, is applied to an Ni-NTA agarose resin column,
after several washings with 5 mM buffer then 50 mM imidazole, the protein MabA, or the derived protein, is eluted with the elution buffer.

44. The process according to claim 43, a recombinant protein MabA or derived recombinant proteins in purified form wherein the different bacteria washing, lysis, washing, and elution buffers, are the following:
bacteria washing buffer: 10 mM potassium phosphate, pH 7.8,
lysis buffer: 50 mM potassium phosphate, pH 7.8 containing 500 mM of NaCl, 5 mM of imidazole,
washing buffer: 50 mM potassium phosphate, pH 7.8 containing 500 mM of NaCl, 5 and 50 mM of imidazole,
elution buffer: 50 mM potassium phosphate, pH 7.8 containing 500 mM of NaCl, and 175 mM of imidazole.

45. The method according to claim 43, wherein the different bacteria washing, lysis, washing, and elution buffers, are the following:
bacteria washing buffer: Tris 10 mM, pH 8.0,
lysis buffer:
50 mM Tris buffer, pH 8.0, supplemented with 300 mM LiSO₄ and 5 mM imidazole;
or 50 mM Tris buffer, pH 8.0, supplemented with 300 mM KCl and 5 mM imidazole,
washing buffer:
50 mM Tris buffer, pH 8.0, supplemented with 300 mM LiSO₄ and 5 or 50 mM imidazole,
or 50 mM Tris buffer, pH 8.0, supplemented with 300 mM KCl and 5 or 50 mM imidazole.
elution buffer:
20 mM MES buffer, pH 6.4, LiSO4 300 mM and 175-750 mM imidazole;
or 20 mM PIPES buffer, pH 8.0, supplemented with 300 mM KCl and 175-750 mM imidazole, 1 mM DTT being added to these buffers in the case of the wild-type protein MabA.

46. Proteins derived from the protein MabA according to claim 41, characterized in that they correspond to the protein MabA the amino acid sequence SEQ ID NO: 1 of which is the following:
MTATATEGAK PPFVSRSVLV TGGNRGIGLA IAQRLAADGH KVAVTHRGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which the cysteine in position 60 is replaced by a valine residue, and/or the glycine in position 139 is replaced by an alanine or a serine, and/or the serine in position 144 is replaced by a leucine residue.

47. Protein derived from the protein MabA according to claim 41, characterized in that it corresponds to the protein MabA in which the cysteine in position 60 is replaced by a valine residue, said derived protein, also called C(60)V, corresponding to the following sequence SEQ ID NO 3:
MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

48. Protein derived from the protein MabA according to claim 41, characterized in that it corresponds to the protein MabA in which the serine in position 144 is replaced by a leucine residue, said derived protein, also called S(144)L, corresponding to the following sequence SEQ ID NO 5:
MTATATEGAK PPFVSRSVLV TGGNRGIGLA IAQRLAADGH KVAVTHRGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGLWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

49. Protein derived from the protein MabA according to claim 41, characterized in that it corresponds to the protein MabA in which the cysteine in position 60 is replaced by a valine residue, and the serine in position 144 is replaced by a leucine residue, said derived protein, also called C (60)V/S(144)L, corresponding to the following sequence SEQ ID NO 7:
MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGLWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH

50. Protein derived from the protein MabA according to claim 41, characterized in that it corresponds to the protein MabA in which the cysteine in position 60 is replaced by a valine residue, the glycine in position 139 is replaced by an alanine or a serine, and the'serine in position 144 is replaced by a leucine residue, said derived protein, also called C(60)V/G(139) [A or S]/S(144)L, corresponding to the following sequence SEQ ID NO 8:
MTATATEGAK PPFVSRSVLV TGGNRGIGLA IAQRLAADGH KVAVTHRGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIXS VSGLWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X represents A or S.

51. Protein MabA corresponding to the sequence SEQ ID NO: 1, or proteins derived from the protein MabA according to claim 6 or proteins corresponding to the sequences SEQ ID NO: 3, 5, 7, or 8, characterized in that they are modified so that they include one or more mutations making it possible to change the specificity of the protein NADPH to NADH.

52. Modified MabA proteins according to claim 51, corresponding to the following sequences:
the sequence SEQ ID NO: 9, corresponding to the sequence SEQ ID NO: 1 comprising the mutations N24D(or E), and/or H46D, namely the following sequence:
MTATATEGAK PPFVSRSVLV TGGX₁RGIGLA IAQRLAADGH KVAVTX₂RCSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X₁ represents D or E, and X₂ represents H or D,
the sequence SEQ ID NO: 10, corresponding to the sequence SEQ ID NO: 3 comprising the mutations N24D(or E), and/or H46D, namely the following sequence:
MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGSWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X₁ represents D or E, and X₂ represents H or D,
the sequence SEQ ID NO: 11, corresponding to the sequence SEQ ID NO: 5 comprising the mutations N24D(or E), and/or H46D, namely the following sequence:
MTATATEGAK PPFVSRSVLV TGGX₁RGIGLA IAQRLAADGH KVAVTX₂RGSG
APKGLFGVEC DVTDSDAVDR AFTAVEEHQG PVEVLVSNAG LSADAFLMRM
TEEKFEKVIN ANLTGAFRVA QRASRSMQRN KFGRMIFIGS VSGLWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X₁ represents D or E, and X₂ represents H or D,
the sequence SEQ ID NO: 12, corresponding to the sequence SEQ ID NO: 7 comprising the mutations N24D(or E), and/or H46D, namely the following sequence:
MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIGS VSGLWCIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X₁ represents D or E, and X₂ represents H or D,
the sequence SEQ ID NO: 13, corresponding to the sequence SEQ ID NO: 8 comprising the mutations N24D(or E), and/or H46D, namely the following sequence:
MTATATEGAK PPFVSRSVLV TGGX₁RGIGLA IAQRLAADGH KVAVTX₂RGSG
APKGLFGVEV DVTDSDAVDR AFTAVEEHQG PVEVLVSNAG LSADAFLMRM
TEEKFEKVIN ANLTGAFRVA QRASRSMQRN KFGRMIFIXS VSGLWGIGNQ
ANYAASKAGV IGMARSIARE LSKANVTANV VAPGYIDTDM TRALDERIQQ
GALQFIPAKR VGTPAEVAGV VSFLASEDAS YISGAVIPVD GGMGMGH
in which X₁ represents D or E, and X₂ represents H or D.

53. Protein MabA corresponding to the sequence SEQ ID NO: 1, or proteins derived from the protein MabA according to claim 46, characterized in that they are modified by insertion, on the N-terminal side, of a poly-histidine tag such as the following sequence SEQ ID NO: 14: MGSSHHHHHH SSGLVPRGSH.

54. Modified proteins MabA according to claim 53, corresponding to the following sequences:
the sequence SEQ ID NO: 15, corresponding to the combination of the sequence SEQ ID NO: 14 and the sequence SEQ ID NO: 1, namely the following sequence:
MGSSHHHHHH SSGLVPRGSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the sequence SEQ ID NO: 16, corresponding to the combination of the sequence SEQ ID NO: 14 and the sequence SEQ ID NO: 3, namely the following sequence:
MGSSHHHHHH SSGLVPRGSH MTATATEGAK PPFVSRSVLV TGGNRGIGLA
IAQRLAADGH KVAVTHRGSG APKGLFCVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the sequence SEQ ID NO: 17, corresponding to the combination of the sequence SEQ ID NO: 14 and the sequence SEQ ID NO: 5, namely the following sequence:
MGSSHHHHHH SSGLVPRGSH MTATATEGAK PPFVSRSVLV TGGNRGIGLA
IAQRLAADGH KVAVTRRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the sequence SEQ ID NO: 18, corresponding to the combination of the sequence SEQ ID NO: 14 and the sequence SEQ ID NO: 7, namely the following sequence:
MGSSHHHHHH SSGLVPRGSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the sequence SEQ ID NO: 19, corresponding to the combination of the sequence SEQ ID NO: 14 and the sequence SEQ ID NO: 9, namely the following sequence:
MGSSHHHHHH SSGLVPRGSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the sequence SEQ ID NO: 20, corresponding to the combination of the sequence SEQ ID NO: 14 and the sequence SEQ ID NO: 10, namely the following sequence:
MGSSHHHHHH SSGLVPRGSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the sequence SEQ ID NO: 21, corresponding to the combination of the sequence SEQ ID NO: 14 and the sequence SEQ ID NO: 11, namely the following sequence:
MGSSHHHHHH SSGLVPRGSH MTATATEGAK PPFVSRSVLV TGGX₁RGIGLA
IAQRLAADGH KVAVTX₂RGSG APKGLFGVEC DVTDSDAVDR AFTAVEERQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the sequence SEQ ID NO: 22, corresponding to the combination of the sequence SEQ ID NO: 14 and the sequence SEQ ID NO: 12, namely the following sequence:
MGSSHHHHHH SSGLVPRGSH MTATATEGAK PPFVSRSVLV TGGX₁RGIGLA
IAQRLAADGH KVAVTX₂RGSC APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the sequence SEQ ID NO: 23, corresponding to the combination of the sequence SEQ ID NO: 14 and the sequence SEQ ID NO: 13, namely the following sequence:
MGSSHHHHHH SSGLVPRGSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIXS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D.

55. Protein MabA corresponding to the sequence SEQ ID NO: 1, or proteins derived from the protein MabA according to claim 46, having an N-terminal GSH sequence, namely the following sequences:
the following sequence SEQ ID NO: 24, corresponding to the combination of the GSH sequence and the sequence SEQ ID NO: 1,
GSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 25, corresponding to the combination of the GSH sequence and the sequence SEQ ID NO: 3,
GSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 26, corresponding to the combination of the GSH sequence and the sequence SEQ ID NO: 5,
GSH MTATATEGAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 27, corresponding to the combination of the GSH sequence and the sequence SEQ ID NO: 7,
GSH MTATATEGAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 28, corresponding to the combination of the GSH sequence and the sequence SEQ ID NO: 9,
GSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 29, corresponding to the combination of the GSH sequence and the sequence SEQ ID NO: 10,
GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 30, corresponding to the combination of the GSH sequence and the sequence SEQ ID NO: 11,
GSH MTATATEGAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 31, corresponding to the combination of the GSH sequence and the sequence SEQ ID NO: 12,
GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 32, corresponding to the combination of the GSH sequence and the sequence SEQ ID NO: 13,
GSH MTATATEGAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIXS VSCLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D.

56. Protein MabA corresponding to the sequence SEQ ID NO: 1, or proteins derived from the protein MabA according to claim 46, in which the first seven amino acids are deleted, namely the following sequences:
the following sequence SEQ ID NO: 33, corresponding to the sequence SEQ ID NO: 1 the first seven amino acids of which are deleted:
GAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 34, corresponding to the sequence SEQ ID NO: 3 the first seven amino acids of which are deleted:
GAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 35, corresponding to the sequence SEQ ID NO: 5 the first seven amino acids of which are deleted:
GAK PPFVSRSVLV TGGNRGIGLA
IAQRLAADGH KVAVTHRGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 36, corresponding to the sequence SEQ ID NO: 7 the first seven amino acids of which are deleted:
GAK PPFVSRSVLV TGGNRGIGLA
IAQRLAADGH KVAVTHRGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
the following sequence SEQ ID NO: 37, corresponding to the sequence SEQ ID NO: 9 the first seven amino acids of which are deleted:
GAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 38, corresponding to the sequence SEQ ID NO: 10 the first seven amino acids of which are deleted:
GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGSWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 39, corresponding to the sequence SEQ ID NO: 11 the first seven amino acids of which are deleted:
OAK PPFVSRSVLV TGGX₁RGIGLA
IAQRLAADGH KVAVTX₂RGSG APKGLFGVEC DVTDSDAVDR AFTAVEEHQG
PVEVLVSNAG LSADAFLMRM TEEKFEKVIN ANLTGAFRVA QRASRSMQRN
KFGRMTFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 40, corresponding to the sequence SEQ ID NO: 12 the first seven amino acids of which are deleted:
GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIGS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D,
the following sequence SEQ ID NO: 41, corresponding to the sequence SEQ ID NO: 13 the first seven amino acids of which are deleted:
GAK PPFVSRSVLV TGGX₁RGIGLA
IAQRLAADGH KVAVTX₂RGSG APKGLFGVEV DVTDSDAVDR AFTAVEEHQG
PVEVLVSNAG LSADAFLMRM TEEKFEKVIN ANLTGAFRVA QRASRSMQRN
KFGRMIFIXS VSGLWGIGNQ ANYAASKAGV IGMARSIARE LSKANVTANV
VAPGYIDTDM TRALDERIQQ GALQFIPAKR VGTPAEVAGV VSFLASEDAS
YISGAVIPVD GGMGMGH
in which X₁ represents D or E, and X₂ represents H or D.

57. Proteins according to claim 41, characterized by having specific enzymatic activity of the substrates of the long-chain type β-ketoacyl.

58. Proteins according to claim 41, the main characteristics of the three-dimensional structure of which, at a resolution of 1.6-2.0 angstroms, detected by X-ray diffraction analysis of the crystals of said proteins, are as represented in FIG. 1 for the recombinant protein MabA corresponding to the sequence SEQ ID NO: 15, in FIG. 2 for the derived protein MabA C(60)V corresponding to the sequence SEQ ID NO: 16, and in FIG. 3 for the derived protein MabA C(60)V/S(144)L corresponding to the sequence SEQ ID NO: 17.

59. Proteins according to claim 41, in crystallized form.

60. Crystals of proteins according to claim 59, as obtained by the hanging-drop vapour diffusion method, by mixing said proteins (1 μl of a 10 mg/ml solution) with a solution of polyethylene glycol, CsCl (150-300 mM), and glycerol (10%) in a buffer (PIPES) at pH 6.2.

61. Crystals of proteins according to claim 59, as obtained according to the crystallization method described in claim 20, said method being carried out from purified proteins.

62. Crystals of the recombinant protein MabA corresponding to the sequence SEQ ID NO: 15 according to claim 59, the atomic coordinates of the three-dimensional structure of which are represented in FIG. 1, and having the following characteristics:
cell parameters:
a=81.403 angströms, b=116.801 angströms, c=52.324 angströms,
α=β=90.00°, γ=122.30°,
space group: C2,
maximum diffraction=2.05 angströms.

63. Crystals of the protein C(60) V corresponding to the sequence SEQ ID NO: 16, according to claim 59, the atomic coordinates of the three-dimensional structure of which are represented in FIG. 2, and having the following characteristics:
cell parameters:
a=82.230 angströms, b=118.610 angströms, c=53.170 angströms,
α=β=90.00°, γ=122.74°,
space group: C2,
maximum diffraction=2.6 angströms.

64. Crystals of the protein C(60)V/S(144) L corresponding to the sequence SEQ ID NO: 18 according to claim 59, the atomic coordinates of the three-dimensional structure of which are represented in FIG. 3, and having the following characteristics:
cell parameters:
a=81.072 angströms, b=117.022 angströms, c=53.170 angströms,
α=β=90.00°, γ=122.42°,
space group: C2,
maximum diffraction=1.75 angströms.

65. Crystals of proteins according to claim 57, in which said proteins are bound to a ligand, namely a molecule capable of binding to the protein MabA.

66. Nucleotide sequence coding for a protein derived from the protein MabA as defined in claim 41.

67. Recombinant nucleotide sequence comprising the nucleotide sequence coding for the protein MabA, or comprising a nucleotide sequence coding for a protein derived from the protein MabA according to claim 41, in combination with the elements necessary for the transcription of this sequence, in particular with a transcription promoter and terminator.

68. Vector, in particular plasmid, containing a nucleotide sequence according to claim 67.

69. Host cells transformed by a vector according to claim 68, said cells being chosen from the bacteria, or any other microorganism used for the production of proteins.

70. Process for the preparation of the recombinant protein MabA in purified form, or of recombinant proteins derived from the protein MabA according to claim 41, characterized in that said process comprises the following stages:
transforming of cells using a recombinant vector,
culturing of the cells thus transformed, and recovery of said proteins produced by said cells, and
purifying said proteins.

71. A method for screening ligands of the protein MabA, comprising the following stages:
being brought into the presence of the recombinant protein MabA in purified form, or a recombinant protein derived from the protein MabA according to claim 41,
detection of any bond between said protein and the ligand tested by measurement, after fluorescence excitation, in particular at 300 nm, of the intensity of fluorescence of said protein emitted between 300 and 400 nm (corresponding essentially to the emission of fluorescence of the single tryptophan W145), and comparison of the intensity of fluorescence emitted in a test in the absence of ligand, the binding of a ligand in the MabA active site being characterized by a quenching of fluorescence.

72. A method for screening ligands inhibiting the protein MabA, comprising the following stages:
being brought into the presence of the recombinant protein MabA in purified form, or a recombinant protein derived from the protein MabA according to claim 41, in a reaction medium comprising a substrate, the coenzyme NADPH and the ligand tested,
detection of a potential inhibiting ability of the ligand tested, by measurement of the enzymatic activity of said protein by kinetic measurement of the absorbance, in particular at 340 nm, and comparison of the gradient of the optical density curve as a function of time with the gradient obtained in a test in the absence of ligand.

73. A method for screening ligands of-the protein MabA, comprising the following stages:
being brought into the presence of the recombinant protein MabA in purified form, or of a recombinant protein derived from the protein MabA according to claim 41, with the ligand tested,
analysis of the three-dimensional structure of the complex formed in soluble phase between said protein and said ligand, in particular by NMR, and by fluorescence.

74. Method for screening ligands of the protein MabA, characterized in that said method comprises the following stages:
co-crystallization of the ligand tested and the recombinant protein MabA in purified form, or of a recombinant protein derived from the protein MabA according to claim 41,
or soaking of the crystals of the protein MabA or of a derived recombinant protein, in optimized solutions containing potential ligands,
analysis of the three-dimensional structure of the abovementioned crystals, in particular by X-ray diffraction (with a view to selecting the ligands having an optimum ability to occupy and block the active site of said proteins).

75. A method for designing or screening ligands of the protein MabA, comprising analyzing the coordinates of the three-dimensional structure of the recombinant protein MabA in purified form, or a recombinant protein derived from the protein MabA according to claim 41, said coordinates being represented in FIGS. 1 to 3, if appropriate in combination with the coordinates of the active site of these proteins.

76. Method for designing or screening, the protein MabA, or proteins with a structure close to the protein MabA, comprising the analyzing the coordinates of a three-dimensional structure of the recombinant protein MabA in purified form, or of a recombinant protein derived from the protein MabA according to claim 41, said coordinates being represented in FIGS. 1 to 3, for screening in silico of the virtual combinatorial libraries of potential ligands, and the detection and rational structural optimization of the molecules capable of binding to said protein.

77. Method of rational design of ligands of the protein MabA, said method being carried out starting with known inhibitors of MabA or inhibitors of proteins homologous to MabA, for which the fine three-dimensional structure of the complex between said inhibitor and the recombinant protein MabA in purified form, or a recombinant protein derived from the protein MabA according to claim 41, was determined, and rational structural optimization of said inhibitors.


SEQUENCE LISTING (41 sequences)

SEQ ID NO: 1 - protein, 247 aa, Mycobacterium tuberculosis
MTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 2 - DNA, 741 bp, Artificial sequence
  Description of the artificial sequence sequence coding for a protein derived from protein MabA of Mycobacterium tuberculosis
atgactgccacagccactgaaggggccaaacccccattcgtatcccgttcagtcctggttaccggaggaaaccgggggatcgggctggcgatcgcacagcggctggctgccgacggccacaaggtggccgtcacccaccgtggatccggagcgccaaaggggctgtttggcgtcgaagttgacgtcaccgacagcgacgccgtcgatcgcgccttcacggcggtagaagagcaccagggtccggtcgaggtgctggtgtccaacgccggcctatccgcggacgcattcctcatgcggatgaccgaggaaaagttcgagaaggtcatcaacgccaacctcaccggggcgttccgggtggctcaacgggcatcgcgcagcatgcagcgcaacaaattcggtcgaatgatattcataggttcggtctccggcagctggggcatcggcaaccaggccaactacgcagcctccaaggccggagtgattggcatggcccgctcgatcgcccgcgagctgtcgaaggcaaacgtgaccgcgaatgtggtggccccgggctacatcgacaccgatatgacccgcgcgctggatgagcggattcagcagggggcgctgcaatttatcccagcgaagcgggtcggcacccccgccgaggtcgccggggtggtcagcttcctggcttccgaggatgcgagctatatctccggtgcggtcatcccggtcgacggcggcatgggtatgggccac

SEQ ID NO: 3 - protein, 247 aa, Artificial sequence
  Description of the artificial sequence sequence coding for a protein derived from protein MabA of Mycobacterium tuberculosis
MTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 4 - DNA, 741 bp, Artificial sequence
  Description of the artificial sequence sequence coding for a protein derived from protein MabA of Mycobacterium tuberculosis
atgactgccacagccactgaaggggccaaacccccattcgtatcccgttcagtcctggttaccggaggaaaccgggggatcgggctggcgatcgcacagcggctggctgccgacggccacaaggtggccgtcacccaccgtggatccggagcgccaaaggggctgtttggcgtcgaatgtgacgtcaccgacagcgacgccgtcgatcgcgccttcacggcggtagaagagcaccagggtccggtcgaggtgctggtgtccaacgccggcctatccgcggacgcattcctcatgcggatgaccgaggaaaagttcgagaaggtcatcaacgccaacctcaccggggcgttccgggtggctcaacgggcatcgcgcagcatgcagcgcaacaaattcggtcgaatgatattcataggttcggtctccggcctctggggcatcggcaaccaggccaactacgcagcctccaaggccggagtgattggcatggcccgctcgatcgcccgcgagctgtcgaaggcaaacgtgaccgcgaatgtggtggccccgggctacatcgacaccgatatgacccgcgcgctggatgagcggattcagcagggggcgctgcaatttatcccagcgaagcgggtcggcacccccgccgaggtcgccggggtggtcagcttcctggcttccgaggatgcgagctatatctccggtgcggtcatcccggtcgacggcggcatgggtatgggccac

SEQ ID NO: 5 - protein, 247 aa, Artificial sequence
  Description of the artificial sequence sequence coding for a protein derived from protein MabA of Mycobacterium tuberculosis
MTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 6 - DNA, 741 bp, Artificial sequence
  Description of the artificial sequence sequence coding for a protein derived from protein MabA of Mycobacterium tuberculosis
atgactgccacagccactgaaggggccaaacccccattcgtatcccgttcagtcctggttaccggaggaaaccgggggatcgggctggcgatcgcacagcggctggctgccgacggccacaaggtggccgtcacccaccgtggatccggagcgccaaaggggctgtttggcgtcgaagttgacgtcaccgacagcgacgccgtcgatcgcgccttcacggcggtagaagagcaccagggtccggtcgaggtgctggtgtccaacgccggcctatccgcggacgcattcctcatgcggatgaccgaggaaaagttcgagaaggtcatcaacgccaacctcaccggggcgttccgggtggctcaacgggcatcgcgcagcatgcagcgcaacaaattcggtcgaatgatattcataggttcggtctccggcctctggggcatcggcaaccaggccaactacgcagcctccaaggccggagtgattggcatggcccgctcgatcgcccgcgagctgtcgaaggcaaacgtgaccgcgaatgtggtggccccgggctacatcgacaccgatatgacccgcgcgctggatgagcggattcagcagggggcgctgcaatttatcccagcgaagcgggtcggcacccccgccgaggtcgccggggtggtcagcttcctggcttccgaggatgcgagctatatctccggtgcggtcatcccggtcgacggcggcatgggtatgggccac

SEQ ID NO: 7 - protein, 247 aa, Artificial sequence
  Description of the artificial sequence sequence coding for a protein derived from protein MabA of Mycobacterium tuberculosis
MTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 8 - protein, 247 aa, Artificial sequence
  Mutant of protein MabA
MTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIXSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 9 - protein, 247 aa, Artificial sequence
  Mutant of protein MabA
MTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 10 - protein, 247 aa, Artificial sequence
  Mutant of protein MabA
MTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 11 - protein, 247 aa, Artificial sequence
  Mutant de la protine de la protine MabA
MTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 12 - protein, 247 aa, Artificial sequence
  Mutant of protein MabA
MTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 13 - protein, 247 aa, Artificial sequence; identical to SEQ ID NO: 12
  Mutant of protein MabA

SEQ ID NO: 14 - protein, 20 aa, Artificial sequence
  Etiquette poly-histidine
MGSSHHHHHHSSGLVPRGSH

SEQ ID NO: 15 - protein, 267 aa, Artificial sequence
  Mutant of protein MabA
MGSSHHHHHHSSGLVPRGSHMTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 16 - protein, 267 aa, Artificial sequence
  Mutant of protein MabA
MGSSHHHHHHSSGLVPRGSHMTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 17 - protein, 267 aa, Artificial sequence
  Mutant of protein MabA
MGSSHHHHHHSSGLVPRGSHMTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 18 - protein, 267 aa, Artificial sequence
  Mutant of protein MabA
MGSSHHHHHHSSGLVPRGSHMTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 19 - protein, 267 aa, Artificial sequence
  Mutant of protein MabA
MGSSHHHHHHSSGLVPRGSHMTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 20 - protein, 267 aa, Artificial sequence
  Mutant of protein MabA
MGSSHHHHHHSSGLVPRGSHMTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 21 - protein, 267 aa, Artificial sequence
  Mutant of protein MabA
MGSSHHHHHHSSGLVPRGSHMTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 22 - protein, 267 aa, Artificial sequence
  Mutant of protein MabA
MGSSHHHHHHSSGLVPRGSHMTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 23 - protein, 267 aa, Artificial sequence; identical to SEQ ID NO: 22
  Mutant of protein MabA

SEQ ID NO: 24 - protein, 250 aa, Artificial sequence
  Mutant of protein MabA
GSHMTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 25 - protein, 250 aa, Artificial sequence
  Mutant of protein MabA
GSHMTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 26 - protein, 250 aa, Artificial sequence
  Mutant of protein MabA
GSHMTATATEGAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 27 - protein, 250 aa, Artificial sequence
  Mutant of protein MabA
GSHMTATATEGAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 28 - protein, 250 aa, Artificial sequence
  Mutant of protein MabA
GSHMTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 29 - protein, 250 aa, Artificial sequence
  Mutant of protein MabA
GSHMTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 30 - protein, 250 aa, Artificial sequence
  Mutant of protein MabA
GSHMTATATEGAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 31 - protein, 250 aa, Artificial sequence
  Mutant of protein MabA
GSHMTATATEGAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 32 - protein, 250 aa, Artificial sequence; identical to SEQ ID NO: 31
  Mutant of protein MabA

SEQ ID NO: 33 - protein, 240 aa, Artificial sequence
  Mutant of protein MabA
GAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 34 - protein, 240 aa, Artificial sequence
  Mutant of protein MabA
GAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 35 - protein, 240 aa, Artificial sequence
  Mutant of protein MabA
GAKPPFVSRSVLVTGGNRGIGLAIAQRLAADGHKVAVTHRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 36 - protein, 240 aa, Artificial sequence
  Mutant of protein MabA
GAKPPFVSRSVLVTGGNRGIGLAIAQRLAADGHKVAVTHRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 37 - protein, 240 aa, Artificial sequence
  Mutant of protein MabA
GAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 38 - protein, 240 aa, Artificial sequence
  Mutant of protein MabA
GAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGSWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 39 - protein, 240 aa, Artificial sequence
  Mutant of protein MabA
GAKPPFVSRSVLVTGGXRGIGLAIAQRLAADGHKVAVTXRGSGAPKGLFGVECDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 40 - protein, 240 aa, Artificial sequence
  Mutant of protein MabA
GAKPPFVSRSVLVTGGXRGIGLAIAQRLAADGHKVAVTXRGSGAPKGLFGVEVDVTDSDAVDRAFTAVEEHQGPVEVLVSNAGLSADAFLMRMTEEKFEKVINANLTGAFRVAQRASRSMQRNKFGRMIFIGSVSGLWGIGNQANYAASKAGVIGMARSIARELSKANVTANVVAPGYIDTDMTRALDERIQQGALQFIPAKRVGTPAEVAGVVSFLASEDASYISGAVIPVDGGMGMGH

SEQ ID NO: 41 - protein, 240 aa, Artificial sequence; identical to SEQ ID NO: 40
  Mutant of protein MabA